SEQUENCE LISTING (84 sequences)

SEQ ID NO: 1 - protein, 317 aa, Homo sapiens
MFQAAERPQEWAMEGPRDGLKKERLLDDRHDSGLDSMKDEEYEQMVKELQEIRLEPQEVPRGSEPWKQQLTEDGDSFLHLAIIHEEKALTMEVIRQVKGDLAFLNFQNNLQQTPLHLAVITNQPEIAEALLGAGCDPELRDFRGNTPLHLACEQGCLASVGVLTQSCTTPHLHSILKATNYNGHTCLHLASIHGYLGIVELLVSLGADVNAQEPCNGRTALHLAVDLQNPDLVSLLLKCGADVNRVTYQGYSPYQLTWGRPSTRIQQQLGQLTLENLQMLPESEDEESYDTESEFTEFTEDELPYDDCVFGGQRLTL

SEQ ID NO: 2 - protein, 356 aa, Homo sapiens
MAGVACLGKAADADEWCDSGLGSLGPDAAAPGGPGLGAELGPGLSWAPLVFGYVTEDGDTALHLAVIHQHEPFLDFLLGFSAGTEYMDLQNDLGQTALHLAAILGETSTVEKLYAAGAGLCVAERRGHTALHLACRVGAHACARALLQPRPRRPREAPDTYLAQGPDRTPDTNHTPVALYPDSDLEKEEEESEEDWKLQLEAENYEGHTPLHVAVIHKDVEMVRLLRDAGADLDKPEPTCGRSPLHLAVEAQAADVLELLLRAGANPAARMYGGRTPLGSAMLRPNPILARLLRAHGAPEPEGEDEKSGPCSSSSDSDSGDEGDEYDDIVVHSSRSQTRLPPTPASKPLPDDPRPV

SEQ ID NO: 3 - protein, 500 aa, Homo sapiens
MNQRRSESRPGNHRLQAYAEPGKGDSGGAGPLSGSARRGRGGGGAIRVRRPCWSGGAGRGGGPAWAVRLPTVTAGWTWPALRTLSSLRAGPSEPHSPGRRPPRAGRPLCQADPQPGKAARRSLEPDPAQTGPRPARAAGMSEARKGPDEAEESQYDSGIESLRSLRSLPESTSAPASGPSDGSPQPCTHPPGPVKEPQEKEDADGERADSTYGSSSLTYTLSLLGGPEAEDPAPRLPLPHVGALSPQQLEALTYISEDGDTLVHLAVIHEAPAVLLCCLALLPQEVLDIQNNLYQTALHLAVHLDQPGAVRALVLKGASRALQDRHGDTALHVACQRQHLACARCLLEGRPEPGRGTSHSLDLQLQNWQGLACLHIATLQKNQPLMELLLRNGADIDVQEGTSGKTALHLAVETQERGLVQFLLQAGAQVDARMLNGCTPLHLAAGRGLMGISSTLCKAGADSLLRNVEDETPQDLTEESLVLLPFDDLKISGKLLLCTD

SEQ ID NO: 4 - protein, 454 aa, Homo sapiens
MPRCPAGAMDEGPVDLRTRPKAAGLPGAALPLRKRPLRAPSPEPAAPRGAAGLVVPLDPLRGGCDLPAVPGPPHGLARPEALYYPGALLPLYPTRAMGSPFPLVNLPTPLYPMMCPMEHPLSADIAMATRADEDGDTPLHIAVVQGNLPAVHRLVNLFQQGGRELDIYNNLRQTPLHLAVITTLPSVVRLLVTAGASPMALDRHGQTAAHLACEHRSPTCLRALLDSAAPGTLDLEARNYDGLTALHVAVNTECQETVQLLLERGADIDAVDIKSGRSPLIHAVENNSLSMVQLLLQHGANVNAQMYSGSSALHSASGRGLLPLVRTLVRSGADSSLKNCHNDTPLMVARSRRVIDILRGKATRPASTSQPDPSPDRSANTSPESSSRLSSNGLLSASPSSSPSQSPPRDPPGFPMAPPNFFLPSPSPPAFLPFAGVLRGPGRPVPPSPAPGGS

SEQ ID NO: 5 - protein, 317 aa, Homo sapiens
MFQAAERPQEWAMEGPRDGLKKERLLDDRHDAGLDAMKDEEYEQMVKELQEIRLEPQEVPRGSEPWKQQLTEDGDSFLHLAIIHEEKALTMEVIRQVKGDLAFLNFQNNLQQTPLHLAVITNQPEIAEALLGAGCDPELRDFRGNTPLHLACEQGCLASVGVLTQSCTTPHLHSILKATNYNGHTCLHLASIHGYLGIVELLVSLGADVNAQEPCNGRTALHLAVDLQNPDLVSLLLKCGADVNRVTYQGYSPYQLTWGRPSTRIQQQLGQLTLENLQMLPESEDEESYDTESEFTEFTEDELPYDDCVFGGQRLTL

SEQ ID NO: 6 - protein, 192 aa, Homo sapiens
MDGSGEQPRGGGPTSSEQIMKTGALLLQGFIQDRAGRMGGEAPELALDPVPQDASTKKLSECLKRIGDELDSNMELQRMIAAVDTDSPREVFFRVAADMFSDGNFNWGRVVALFYFASKLVLKALCTKVPELIRTIMGWTLDFLRERLLGWIQDQGGWDGLLSYFGTPTWQTVTIFVAGVLTASLTIWKKMG

SEQ ID NO: 7 - protein, 199 aa, Homo sapiens
MSSGNAKIGHPAPNFKATAVMPDGQFKDISLSDYKGKYVVFFFYPLDFTFVCPTEIIAFSDRAEEFKKLNCQVIGASVDSHFCHLAWVNTPKKQGGLGPMNIPLVSDPKRTIAQDYGVLKADEGISFRGLFIIDDKGILRQITVNDLPVGRSVDETLRLVQAFQFTDKHGEVCPAGWKPGSDTIKPDVQKSKEYFSKQK

SEQ ID NO: 8 - protein, 198 aa, Homo sapiens
MASGNARIGKPAPDFKATAVVDGAFKEVKLSDYKGKYVVLFFYPLDFTFVCPTEIIAFSNRAEDFRKLGCEVLGVSVDSQFTHLAWINTPRKEGGLGPLNIPLLADVTRRLSEDYGVLKTDEGIAYRGLFIIDGKGVLRQITVNDLPVGRSVDEALRLVQAFQYTDEHGEVCPAGWKPGSDTIKPNVDDSKEYFSKHN

SEQ ID NO: 9 - protein, 343 aa, Bacteriophage P1
MSNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGD

SEQ ID NO: 10 - protein, 469 aa, Streptococcus pyogenes
MHSFPPLLLLLFWGVVSHSFPATLETQEQDVDLVQKYLEKYYNLKNDGRQVEKRRNSGPVVEKLKQMQEFFGLKVTGKPDAETLKVMKQPRCGVPDVAQFVLTEGNPRWEQTHLTYRIENYTPDLPRADVDHAIEKAFQLWSNVTPLTFTKVSEGQADIMISFVRGDHRDNSPFDGPGGNLAHAFQPGPGIGGDAHFDEDERWTNNFREYNLHRVAAHELGHSLGLSHSTDIGALMYPSYTFSGDVQLAQDDIDGIQAIYGRSQNPVQPIGPQTPKACDSKLTFDAITTIRGEVMFFKDRFYMRTNPFYPEVELNFISVFWPQLPNGLEAAYEFADRDEVRFFKGNKYWAVQGQNVLHGYPKDIYSSFGFPRTVKHIDAALSEENTGKTYFFVANKYWRYDEYKRSMDPGYPKMIAHDFPGIGHKVDAVFMKDGFFYFFHGTRQYKFDPKTKRILTLQKANSWFNCRKN

SEQ ID NO: 11 - protein, 1300 aa, Francisella tularensis
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 12 - protein, 536 aa, Homo sapiens
MEFSSPSREECPKPLSRVSIMAGSLTGLLLLQAVSWASGARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 13 - protein, 469 aa, Homo sapiens; identical to SEQ ID NO: 10

SEQ ID NO: 14 - protein, 207 aa, Homo sapiens
MAPFEPLASGILLLLWLIAPSRACTCVPPHPQTAFCNSDLVIRAKFVGTPEVNQTTLYQRYEIKMTKMYKGFQALGDAADIRFVYTPAMESVCGYFHRSHNRSEEFLIAGKLQDGLLHITTCSFVAPWNSLSLAQRRGFTKTYTVGCEECTVFPCLSIPCKLQSGTHCLWTDQLLQGSEKGFQSRHLACLPREPGLCTWQSLRSQIA

SEQ ID NO: 15 - protein, 220 aa, Homo sapiens
MGAAARTLRLALGLLLLATLLRPADACSCSPVHPQQAFCNADVVIRAKAVSEKEVDSGNDIYGNPIKRIQYEIKQIKMFKGPEKDIEFIYTAPSSAVCGVSLDVGGKKEYLIAGKAEGDGKMHITLCDFIVPWDTLSTTQKKSLNHRYQMGCECKITRCPMIPCYISSPDECLWMDWVTEKNINGHQAKFFACIKRSDGSCAWYRGAAPPKQEFLDIEDP

SEQ ID NO: 16 - protein, 211 aa, Homo sapiens
MTPWLGLIVLLGSWSLGDWGAEACTCSPSHPQDAFCNSDIVIRAKVVGKKLVKEGPFGTLVYTIKQMKMYRGFTKMPHVQYIHTEASESLCGLKLEVNKYQYLLTGRVYDGKMYTGLCNFVERWDQLTLSQRKGLNYRYHLGCNCKIKSCYYLPCFVTSKNECLWTDMLSNFGYPGYQSKHYACIRQKGGYCSWYRGWAPPDKSIINATDP

SEQ ID NO: 17 - protein, 224 aa, Homo sapiens
MPGSPRPAPSWVLLLRLLALLRPPGLGEACSCAPAHPQQHICHSALVIRAKISSEKVVPASADPADTEKMLRYEIKQIKMFKGFEKVKDVQYIYTPFDSSLCGVKLEANSQKQYLLTGQVLSDGKVFIHLCNYIEPWEDLSLVQRESLNHHYHLNCGCQITTCYTVPCTISAPNECLWTDWLLERKLYGYQAQHYVCMKHVDGTCSWYRGHLPLRKEFVDIVQP

SEQ ID NO: 18 - protein, 404 aa, Homo sapiens
MADKVLKEKRKLFIRSMGEGTINGLLDELLQTRVLNKEEMEKVKRENATVMDKTRALIDSVIPKGAQACQICITYICEEDSYLAGTLGLSADQTSGNYLNMQDSQGVLSSFPAPQAVQDNPAMPTSSGSEGNVKLCSLEEAQRIWKQKSAEIYPIMDKSSRTRLALIICNEEFDSIPRRTGAEVDITGMTMLLQNLGYSVDVKKNLTASDMTTELEAFAHRPEHKTSDSTFLVFMSHGIREGICGKKHSEQVPDILQLNAIFNMLNTKNCPSLKDKPKVIIIQACRGDSPGVVWFKDSVGVSGNLSLPTTEEFEDDAIKKAHIEKDFIAFCSSTPDNVSWRHPTMGSVFIGRLIEHMQEYACSCDVEEIFRKVRFSFEQPDGRAQMPTTERVTLTRCFYLFPGH

SEQ ID NO: 19 - protein, 452 aa, Homo sapiens
MAAPSAGSWSTFQHKELMAADRGRRILGVCGMHPHHQETLKKNRVVLAKQLLLSELLEHLLEKDIITLEMRELIQAKVGSFSQNVELLNLLPKRGPQAFDAFCEALRETKQGHLEDMLLTTLSGLQHVLPPLSCDYDLSLPFPVCESCPLYKKLRLSTDTVEHSLDNKDGPVCLQVKPCTPEFYQTHFQLAYRLQSRPRGLALVLSNVHFTGEKELEFRSGGDVDHSTLVTLFKLLGYDVHVLCDQTAQEMQEKLQNFAQLPAHRVTDSCIVALLSHGVEGAIYGVDGKLLQLQEVFQLFDNANCPSLQNKPKMFFIQACRGDETDRGVDQQDGKNHAGSPGCEESDAGKEKLPKMRLPTRSDMICGYACLKGTAAMRNTKRGSWYIEALAQVFSERACDMHVADMLVKVNALIKDREGYAPGTEFHRCKEMSEYCSTLCRHLYLFPGHPPT

SEQ ID NO: 20 - protein, 277 aa, Homo sapiens
MENTENSVDSKSIKNLEPKIIHGSESMDSGISLDNSYKMDYPEMGLCIIINNKNFHKSTGMTSRSGTDVDAANLRETFRNLKYEVRNKNDLTREEIVELMRDVSKEDHSKRSSFVCVLLSHGEEGIIFGTNGPVDLKKITNFFRGDRCRSLTGKPKLFIIQACRGTELDCGIETDSGVDDDMACHKIPVEADFLYAYSTAPGYYSWRNSKDGSWFIQSLCAMLKQYADKLEFMHILTRVNRKVATEFESFSFDATFHAKKQIPCIVSMLTKELYFYH

SEQ ID NO: 21 - protein, 377 aa, Homo sapiens
MAEGNHRKKPLKVLESLGKDFLTGVLDNLVEQNVLNWKEEEKKKYYDAKTEDKVRVMADSMQEKQRMAGQMLLQTFFNIDQISPNKKAHPNMEAGPPESGESTDALKLCPHEEFLRLCKERAEEIYPIKERNNRTRLALIICNTEFDHLPPRNGADFDITGMKELLEGLDYSVDVEENLTARDMESALRAFATRPEHKSSDSTFLVLMSHGILEGICGTVHDEKKPDVLLYDTIFQIFNNRNCLSLKDKPKVIIVQACRGANRGELWVRDSPASLEVASSQSSENLEEDAVYKTHVEKDFIAFCSSTPHNVSWRDSTMGSIFITQLITCFQKYSWCCHLEEVFRKVQQSFETPRAKAQMPTIERLSMTRYFYLFPGN

SEQ ID NO: 22 - protein, 434 aa, Homo sapiens
MAEDSGKKKRRKNFEAMFKGILQSGLDNFVINHMLKNNVAGQTSIQTLVPNTDQKSTSVKKDNHKKKTVKMLEYLGKDVLHGVFNYLAKHDVLTLKEEEKKKYYDTKIEDKALILVDSLRKNRVAHQMFTQTLLNMDQKITSVKPLLQIEAGPPESAESTNILKLCPREEFLRLCKKNHDEIYPIKKREDRRRLALIICNTKFDHLPARNGAHYDIVGMKRLLQGLGYTVVDEKNLTARDMESVLRAFAARPEHKSSDSTFLVLMSHGILEGICGTAHKKKKPDVLLYDTIFQIFNNRNCLSLKDKPKVIIVQACRGEKHGELWVRDSPASLALISSQSSENLEADSVCKIHEEKDFIAFCSSTPHNVSWRDRTRGSIFITELITCFQKYSCCCHLMEIFRKVQKSFEVPQAKAQMPTIERATLTRDFYLFPGN

SEQ ID NO: 23 - protein, 293 aa, Homo sapiens
MSSASGLRRGHPAGGEENMTETDAFYKREMFDPAEKYKMDHRRRGIALIFNHERFFWHLTLPERRGTCADRDNLTRRFSDLGFEVKCFNDLKAEELLLKIHEVSTVSHADADCFVCVFLSHGEGNHIYAYDAKIEIQTLTGLFKGDKCHSLVGKPKIFIIQACRGNQHDVPVIPLDVVDNQTEKLDTNITEVDAASVYTLPAGADFLMCYSVAEGYYSHRETVNGSWYIQDLCEMLGKYGSSLEFTELLTLVNRKVSQRRVDFCKDPSAIGKKQVPCFASMLTKKLHFFPKSN

SEQ ID NO: 24 - protein, 303 aa, Homo sapiens
MADDQGCIEEQGVEDSANEDSVDAKPDRSSFVPSLFSKKKKNVTMRSIKTTRDRVPTYQYNMNFEKLGKCIIINNKNFDKVTGMGVRNGTDKDAEALFKCFRSLGFDVIVYNDCSCAKMQDLLKKASEEDHTNAACFACILLSHGEENVIYGKDGVTPIKDLTAHFRGDRCKTLLEKPKLFFIQACRGTELDDGIQADSGPINDTDANPRYKIPVEADFLFAYSTVPGYYSWRSPGRGSWFVQALCSILEEHGKDLEIMQILTRVNDRVARHFESQSDDPHFHEKKQIPCVVSMLTKELYFSQ

SEQ ID NO: 25 - protein, 479 aa, Homo sapiens
MDFSRNLYDIGEQLDSEDLASLKFLSLDYIPQRKQEPIKDALMLFQRLQEKRMLEESNLSFLKELLFRINRLDLLITYLNTRKEEMERELQTPGRAQISAYRVMLYQISEEVSRSELRSFKFLLQEEISKCKLDDDMNLLDIFIEMEKRVILGEGKLDILKRVCAQINKSLLKIINDYEEFSKERSSSLEGSPDEFSNGEELCGVMTISDSPREQDSESQTLDKVYQMKSKPRGYCLIINNHNFAKAREKVPKLHSIRDRNGTHLDAGALTTTFEELHFEIKPHDDCTVEQIYEILKIYQLMDHSNMDCFICCILSHGDKGIIYGTDGQEAPIYELTSQFTGLKCPSLAGKPKVFFIQACQGDNYQKGIPVETDSEEQPYLEMDLSSPQTRYIPDEADFLLGMATVNNCVSYRNPAEGTWYIQSLCQSLRERCPRGDDILTILTEVNYEVSNKDDKKNMGKQMPQPTFTLRKKLVFPSD

SEQ ID NO: 26 - protein, 416 aa, Homo sapiens
MDEADRRLLRRCRLRLVEELQVDQLWDALLSRELFRPHMIEDIQRAGSGSRRDQARQLIIDLETRGSQALPLFISCLEDTGQDMLASFLRTNRQAAKLSKPTLENLTPVVLRPEIRKPEVLRPETPRPVDIGSGGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 27 - protein, 521 aa, Homo sapiens
MKSQGQHWYSSSDKNCKVSFREKLLIIDSNLGVQDVENLKFLCIGLVPNKKLEKSSSASDVFEHLLAEDLLSEEDPFFLAELLYIIRQKKLLQHLNCTKEEVERLLPTRQRVSLFRNLLYELSEGIDSENLKDMIFLLKDSLPKTEMTSLSFLAFLEKQGKIDEDNLTCLEDLCKTVVPKLLRNIEKYKREKAIQIVTPPVDKEAESYQGEEELVSQTDVKTFLEALPQESWQNKHAGSNGNRATNGAPSLVSRGMQGASANTLNSETSTKRAAVYRMNRNHRGLCVIVNNHSFTSLKDRQGTHKDAEILSHVFQWLGFTVHIHNNVTKVEMEMVLQKQKCNPAHADGDCFVFCILTHGRFGAVYSSDEALIPIREIMSHFTALQCPRLAEKPKLFFIQACQGEEIQPSVSIEADALNPEQAPTSLQDSIPAEADFLLGLATVPGYVSFRHVEEGSWYIQSLCNHLKKLVPRMLKFLEKTMEIRGRKRTVWGAKQISATSLPTAISAQTPRPPMRRWSSVS

SEQ ID NO: 28 - protein, 373 aa, Homo sapiens
MAENKHPDKPLKVLEQLGKEVLTEYLEKLVQSNVLKLKEEDKQKFNNAERSDKRWVFVDAMKKKHSKVGEMLLQTFFSVDPGSHHGEANLEMEEPEESLNTLKLCSPEEFTRLCREKTQEIYPIKEANGRTRKALIICNTEFKHLSLRYGANFDIIGMKGLLEDLGYDVVVKEELTAEGMESEMKDFAALSEHQTSDSTFLVLMSHGTLHGICGTMHSEKTPDVLQYDTIYQIFNNCHCPGLRDKPKVIIVQACRGGNSGEMWIRESSKPQLCRGVDLPRNMEADAVKLSHVEKDFIAFYSTTPHHLSYRDKTGGSYFITRLISCFRKHACSCHLFDIFLKVQQSFEKASIHSQMPTIDRATLTRYFYLFPGN

SEQ ID NO: 29 - protein, 341 aa, Homo sapiens
MADEKPSNGVLVHMVKLLIKTFLDGIFDDLMENNVLNTDEIHLIGKCLKFVVSNAENLVDDITETAQTAGKIFREHLWNSKKQLSSDISSDGEREANMPGLNIRNKEFNYLHNRNGSELDLLGMRDLLENLGYSVVIKENLTAQEMETALRQFAAHPEHQSSDSTFLVFMSHSILNGICGTKHWDQEPDVLHDDTIFEIFNNRNCQSLKDKPKVIIMQACRGNGAGIVWFTTDSGKAGADTHGRLLQGNICNDAVTKAHVEKDFIAFKSSTPHNVSWRHETNGSVFISQIIYYFREYSWSHHLEEIFQKVQHSFETPNILTQLPTIERLSMTRYFYLFPGN

SEQ ID NO: 30 - protein, 377 aa, Homo sapiens
MAEDKHNKNPLKMLESLGKELISGLLDDFVEKNVLKLEEEEKKKIYDAKLQDKARVLVDSIRQKNQEAGQVFVQTFLNIDKNSTSIKAPEETVAGPDESVGSAATLKLCPHEEFLKLCKERAGEIYPIKERKDRTRLALIICNTEFDHMPPRNGAALDILGMKQLLEGLGYTVEVEEKLTARDMESVLWKFAAREEHKSSDSTFLVFMSHGILDGICGTMHSEEEPDVLPYDTIFRTFNNRNCLSLKDKPKVIIVQACRGANRGELWVSDSPPALADSFSQSSENLEEDAVYKTHVEKDFIAFCSSTPHNVSWRDIKKGSLFITRLITCFQKYAWCCHLEEVFRKVQQSFEKPNVKAQMPTVERLSMTRYFYLFPGN

SEQ ID NO: 31 - protein, 242 aa, Homo sapiens
MSNPRSLEEEKYDMSGARLALILCVTKAREGSEEDLDALEHMFRQLRFESTMKRDPTAEQFQEELEKFQQAIDSREDPVSCAFVVLMAHGREGFLKGEDGEMVKLENLFEALNNKNCQALRAKPKVYIIQACRGEQRDPGETVGGDEIVMVIKDSPQTIPTYTDALHVYSTVEGYIAYRHDQKGSCFIQTLVDVFTKRKGHILELLTEVTRRMAEAELVQEGKARKTNPEIQSTLRKRLYLQ

SEQ ID NO: 32 - protein, 480 aa, Homo sapiens
MIRAAPPPLFLLLLLLLLLVSWASRGEAAPDQDEIQRLPGLAKQPSFRQYSGYLKGSGSKHLHYWFVESQKDPENSPVVLWLNGGPGCSSLDGLLTEHGPFLVQPDGVTLEYNPYSWNLIANVLYLESPAGVGFSYSDDKFYATNDTEVAQSNFEALQDFFRLFPEYKNNKLFLTGESYAGIYIPTLAVLVMQDPSMNLQGLAVGNGLSSYEQNDNSLVYFAYYHGLLGNRLWSSLQTHCCSQNKCNFYDNKDLECVTNLQEVARIVGNSGLNIYNLYAPCAGGVPSHFRYEKDTVVVQDLGNIFTRLPLKRMWHQALLRSGDKVRMDPPCTNTTAASTYLNNPYVRKALNIPEQLPQWDMCNFLVNLQYRRLYRSMNSQYLKLLSSQKYQILLYNGDVDMACNFMGDEWFVDSLNQKMEVQRRPWLVKYGDSGEQIAGFVKEFSHIAFLTIKGAGHMVPTDKPLAAFTMFSRFLNKQPY

SEQ ID NO: 33 - protein, 339 aa, Homo sapiens
MWQLWASLCCLLVLANARSRPSFHPLSDELVNYVNKRNTTWQAGHNFYNVDMSYLKRLCGTFLGGPKPPQRVMFTEDLKLPASFDAREQWPQCPTIKEIRDQGSCGSCWAFGAVEAISDRICIHTNAHVSVEVSAEDLLTCCGSMCGDGCNGGYPAEAWNFWTRKGLVSGGLYESHVGCRPYSIPPCEHHVNGSRPPCTGEGDTPKCSKICEPGYSPTYKQDKHYGYNSYSVSNSEKDIMAEIYKNGPVEGAFSVYSDFLLYKSGVYQHVTGEMMGGHAIRILGWGVENGTPYWLVANSWNTDWGDNGFFKILRGQDHCGIESEVVAGIPRTDQYWEKI

SEQ ID NO: 34 - protein, 463 aa, Homo sapiens
MGAGPSLLLAALLLLLSGDGAVRCDTPANCTYLDLLGTWVFQVGSSGSQRDVNCSVMGPQEKKVVVYLQKLDTAYDDLGNSGHFTIIYNQGFEIVLNDYKWFAFFKYKEEGSKVTTYCNETMTGWVHDVLGRNWACFTGKKVGTASENVYVNIAHLKNSQEKYSNRLYKYDHNFVKAINAIQKSWTATTYMEYETLTLGDMIRRSGGHSRKIPRPKPAPLTAEIQQKILHLPTSWDWRNVHGINFVSPVRNQASCGSCYSFASMGMLEARIRILTNNSQTPILSPQEVVSCSQYAQGCEGGFPYLIAGKYAQDFGLVEEACFPYTGTDSPCKMKEDCFRYYSSEYHYVGGFYGGCNEALMKLELVHHGPMAVAFEVYDDFLHYKKGIYHHTGLRDPFNPFELTNHAVLLVGYGTDSASGMDYWIVKNSWGTGWGENGYFRIRRGTDECAIESIAVAATPIPKL

SEQ ID NO: 35 - protein, 412 aa, Homo sapiens
MQPSSLLPLALCLLAAPASALVRIPLHKFTSIRRTMSEVGGSVEDLIAKGPVSKYSQAVPAVTEGPIPEVLKNYMDAQYYGEIGIGTPPQCFTVVFDTGSSNLWVPSIHCKLLDIACWIHHKYNSDKSSTYVKNGTSFDIHYGSGSLSGYLSQDTVSVPCQSASSASALGGVKVERQVFGEATKQPGITFIAAKFDGILGMAYPRISVNNVLPVFDNLMQQKLVDQNIFSFYLSRDPDAQPGGELMLGGTDSKYYKGSLSYLNVTRKAYWQVHLDQVEVASGLTLCKEGCEAIVDTGTSLMVGPVDEVRELQKAIGAVPLIQGEYMIPCEKVSTLPAITLKLGGKGYKLSPEDYTLKVSQAGKTLCLSGFMGMDIPPPSGPLWILGDVFIGRYYTVFDRDNNRVGFAEAARL

SEQ ID NO: 36 - protein, 401 aa, Homo sapiens
MKTLLLLLLVLLELGEAQGSLHRVPLRRHPSLKKKLRARSQLSEFWKSHNLDMIQFTESCSMDQSAKEPLINYLDMEYFGTISIGSPPQNFTVIFDTGSSNLWVPSVYCTSPACKTHSRFQPSQSSTYSQPGQSFSIQYGTGSLSGIIGADQVSAFATQVEGLTVVGQQFGESVTEPGQTFVDAEFDGILGLGYPSLAVGGVTPVFDNMMAQNLVDLPMFSVYMSSNPEGGAGSELIFGGYDHSHFSGSLNWVPVTKQAYWQIALDNIQVGGTVMFCSEGCQAIVDTGTSLITGPSDKIKQLQNAIGAAPVDGEYAVECANLNVMPDVTFTINGVPYTLSPTAYTLLDFVDGMQFCSSGFQGLDIHPPAGPLWILGDVFIRQFYSVFDRGNNRVGLAPAVP

SEQ ID NO: 37 - protein, 484 aa, Homo sapiens [sequence omitted]

SEQ ID NO: 38 - protein, 255 aa, Homo sapiens
MQPLLLLLAFLLPTGAEAGEIIGGRESRPHSRPYMAYLQIQSPAGQSRCGGFLVREDFVLTAAHCWGSNINVTLGAHNIQRRENTQQHITARRAIRHPQYNQRTIQNDIMLLQLSRRVRRNRNVNPVALPRAQEGLRPGTLCTVAGWGRVSMRRGTDTLREVQLRVQRDRQCLRIFGSYDPRRQICVGDRRERKAAFKGDSGGPLLCNNVAHGIVSYGKSSGVPPEVFTRVSSFLPWIRTTMRSFKLLDQMETPL

SEQ ID NO: 39 - protein, 335 aa, Homo sapiens
MWATLPLLCAGAWLLGVPVCGAAELCVNSLEKFHFKSWMSKHRKTYSTEEYHHRLQTFASNWRKINAHNNGNHTFKMALNQFSDMSFAEIKHKYLWSEPQNCSATKSNYLRGTGPYPPSVDWRKKGNFVSPVKNQGACGSCWTFSTTGALESAIAIATGKMLSLAEQQLVDCAQDFNNHGCQGGLPSQAFEYILYNKGIMGEDTYPYQGKDGYCKFQPGKAIGFVKDVANITIYDEEAMVEAVALYNPVSFAFEVTQDFMMYRTGIYSSTSCHKTPDKVNHAVLAVGYGEKNGIPYWIVKNSWGPQWGMNGYFLIERGKNMCGLAACASYPIPLV

SEQ ID NO: 40 - protein, 329 aa, Homo sapiens
MWGLKVLLLPVVSFALYPEEILDTHWELWKKTHRKQYNNKVDEISRRLIWEKNLKYISIHNLEASLGVHTYELAMNHLGDMTSEEVVQKMTGLKVPLSHSRSNDTLYIPEWEGRAPDSVDYRKKGYVTPVKNQGQCGSCWAFSSVGALEGQLKKKTGKLLNLSPQNLVDCVSENDGCGGGYMTNAFQYVQKNRGIDSEDAYPYVGQEESCMYNPTGKAAKCRGYREIPEGNEKALKRAVARVGPVSVAIDASLTSFQFYSKGVYYDESCNSDNLNHAVLAVGYGIQKGNKHWIIKNSWGENWGNKGYILMARNKNNACGIANLASFPKM

SEQ ID NO: 41 - protein, 333 aa, Homo sapiens [sequence omitted]

SEQ ID NO: 42 - protein, 334 aa, Homo sapiens
MNLSLVLAAFCLGIASAVPKFDQNLDTKWYQWKATHRRLYGANEEGWRRAVWEKNMKMIELHNGEYSQGKHGFTMAMNAFGDMTNEEFRQMMGCFRNQKFRKGKVFREPLFLDLPKSVDWRKKGYVTPVKNQKQCGSCWAFSATGALEGQMFRKTGKLVSLSEQNLVDCSRPQGNQGCNGGFMARAFQYVKENGGLDSEESYPYVAVDEICKYRPENSVANDTGFTVVAPGKEKALMKAVATVGPISVAMDAGHSSFQFYKSGIYFEPDCSSKNLDHGVLVVGYGFEGANSNNSKYWLVKNSWGPEWGSNGYVKIAKDKNNHCGIATAASYPNV

SEQ ID NO: 43 - protein, 321 aa, Homo sapiens
MDVRALPWLPWLLWLLCRGGGDADSRAPFTPTWPRSREREAAAFRESLNRHRYLNSLFPSENSTAFYGINQFSYLFPEEFKAIYLRSKPSKFPRYSAEVHMSIPNVSLPLRFDWRDKQVVTQVRNQQMCGGCWAFSVVGAVESAYAIKGKPLEDLSVQQVIDCSYNNYGCNGGSTLNALNWLNKMQVKLVKDSEYPFKAQNGLCHYFSGSHSGFSIKGYSAYDFSDQEDEMAKALLTFGPLVVIVDAVSWQDYLGGIIQHHCSSGEANHAVLITGFDKTGSTPYWIVRNSWGSSWGVDGYAHVKMGSNVCGIADSVSSIFV

SEQ ID NO: 44 - protein, 331 aa, Homo sapiens
MKRLVCVLLVCSSAVAQLHKDPTLDHHWHLWKKTYGKQYKEKNEEAVRRLIWEKNLKFVMLHNLEHSMGMHSYDLGMNHLGDMTSEEVMSLMSSLRVPSQWQRNITYKSNPNRILPDSVDWREKGCVTEVKYQGSCGACWAFSAVGALEAQLKLKTGKLVSLSAQNLVDCSTEKYGNKGCNGGFMTTAFQYIIDNKGIDSDASYPYKAMDQKCQYDSKYRAATCSKYTELPYGREDVLKEAVANKGPVSVGVDARHPSFFLYRSGVYYEPSCTQNVNHGVLVVGYGDLNGKEYWLVKNSWGHNFGEEGYIRMARNKGNHCGIASFPSYPEI

SEQ ID NO: 45 - protein, 376 aa, Homo sapiens
MALTAHPSCLLALLVAGLAQGIRGPLRAQDLGPQPLELKEAFKLFQIQFNRSYLSPEEHAHRLDIFAHNLAQAQRLQEEDLGTAEFGVTPFSDLTEEEFGQLYGYRRAAGGVPSMGREIRSEEPEESVPFSCDWRKVASAISPIKDQKNCNCCWAMAAAGNIETLWRISFWDFVDVSVQELLDCGRCGDGCHGGFVWDAFITVLNNSGLASEKDYPFQGKVRAHRCHPKKYQKVAWIQDFIMLQNNEHRIAQYLATYGPITVTINMKPLQLYRKGVIKATPTTCDPQLVDHSVLLVGFGSVKSEEGIWAETVSSQSQPQPPHPTPYWILKNSWGAQWGEKGYFRLHRGSNTCGITKFPLTARVQKPDMKPRVSCPP

SEQ ID NO: 46 - protein, 303 aa, Homo sapiens
MARRGPGWRPLLLLVLLAGAAQGGLYFRRGQTCYRPLRGDGLAPLGRSTYPRPHEYLSPADLPKSWDWRNVDGVNYASITRNQHIPQYCGSCWAHASTSAMADRINIKRKGAWPSTLLSVQNVIDCGNAGSCEGGNDLSVWDYAHQHGIPDETCNNYQAKDQECDKFNQCGTCNEFKECHAIRNYTLWRVGDYGSLSGREKMMAEIYANGPISCGIMATERLANYTGGIYAEYQDTTYINHVVSVAGWGISDGTEYWIVRNSWGEPWGERGWLRIVTSTYKDGKGARYNLAIEEHCTFGDPIV

SEQ ID NO: 47 - protein, 338 aa, Homo sapiens
MLQKPKSVKLRALRSPRKFGVAGRSCQEVLRKGCLRFQLPERGSRLCLYEDGTELTEDYFPSVPDNAELVLLTLGQAWQGYVSDIRRFLSAFHEPQVGLIQAAQQLLCDEQAPQRQRLLADLLHNVSQNIAAETRAEDPPWFEGLESRFQSKSGYLRYSCESRIRSYLREVSSYPSTVGAEAQEEFLRVLGSMCQRLRSMQYNGSYFDRGAKGGSRLCTPEGWFSCQGPFDMDSCLSRHSINPYSNRESRILFSTWNLDHIIEKKRTIIPTLVEAIKEQDGREVDWEYFYGLLFTSENLKLVHIVCHKKTTHKLNCDPSRIYKPQTRLKRKQPVRKRQ

SEQ ID NO: 48 - protein, 360 aa, Homo sapiens
MSQERPTFYRQELNKTIWEVPERYQNLSPVGSGAYGSVCAAFDTKTGLRVAVKKLSRPFQSIIHAKRTYRELRLLKHMKHENVIGLLDVFTPARSLEEFNDVYLVTHLMGADLNNIVKCQKLTDDHVQFLIYQILRGLKYIHSADIIHRDLKPSNLAVNEDCELKILDFGLARHTDDEMTGYVATRWYRAPEIMLNWMHYNQTVDIWSVGCIMAELLTGRTLFPGTDHIDQLKLILRLVGTPGAELLKKISSESARNYIQSLTQMPKMNFANVFIGANPLAVDLLEKMLVLDSDKRITAAQALAHAYFAQYHDPDDEPVADPYDQSFESRDLLIDEWKSLTYDEVISFVPPPLDQEEMES

SEQ ID NO: 49 - protein, 364 aa, Homo sapiens
MSGPRAGFYRQELNKTVWEVPQRLQGLRPVGSGAYGSVCSAYDARLRQKVAVKKLSRPFQSLIHARRTYRELRLLKHLKHENVIGLLDVFTPATSIEDFSEVYLVTTLMGADLNNIVKCQALSDEHVQFLVYQLLRGLKYIHSAGIIHRDLKPSNVAVNEDCELRILDFGLARQADEEMTGYVATRWYRAPEIMLNWMHYNQTVDIWSVGCIMAELLQGKALFPGSDYIDQLKRIMEVVGTPSPEVLAKISSEHARTYIQSLPPMPQKDLSSIFRGANPLAIDLLGRMLVLDSDQRVSAAEALAHAYFSQYHDPEDEPEAEPYDESVEAKERTLEEWKELTYQEVLSFKPPEPPKPPGSLEIEQ

SEQ ID NO: 50 - protein, 367 aa, Homo sapiens
MSSPPPARSGFYRQEVTKTAWEVRAVYRDLQPVGSGAYGAVCSAVDGRTGAKVAIKKLYRPFQSELFAKRAYRELRLLKHMRHENVIGLLDVFTPDETLDDFTDFYLVMPFMGTDLGKLMKHEKLGEDRIQFLVYQMLKGLRYIHAAGIIHRDLKPGNLAVNEDCELKILDFGLARQADSEMTGYVVTRWYRAPEVILNWMRYTQTVDIWSVGCIMAEMITGKTLFKGSDHLDQLKEIMKVTGTPPAEFVQRLQSDEAKNYMKGLPELEKKDFASILTNASPLAVNLLEKMLVLDAEQRVTAGEALAHPYFESLHDTEDEPQVQKYDDSFDDVDRTLDEWKRVTYKEVLSFKPPRQLGARVSKETPL

SEQ ID NO: 51 - protein, 365 aa, Homo sapiens
MSLIRKKGFYKQDVNKTAWELPKTYVSPTHVGSGAYGSVCSAIDKRSGEKVAIKKLSRPFQSEIFAKRAYRELLLLKHMQHENVIGLLDVFTPASSLRNFYDFYLVMPFMQTDLQKIMGMEFSEEKIQYLVYQMLKGLKYIHSAGVVHRDLKPGNLAVNEDCELKILDFGLARHADAEMTGYVVTRWYRAPEVILSWMHYNQTVDIWSVGCIMAEMLTGKTLFKGKDYLDQLTQILKVTGVPGTEFVQKLNDKAAKSYIQSLPQTPRKDFTQLFPRASPQAADLLEKMLELDVDKRLTAAQALTHPFFEPFRDPEEETEAQQPFDDSLEHEKLTVDEWKQHIYKEIVNFSPIARKDSRRRSGMKL

SEQ ID NO: 52 - protein, 403 aa, Homo sapiens [sequence omitted]

SEQ ID NO: 53 - protein, 1154 aa, Homo sapiens
MQYLNIKEDCNAMAFCAKMRSSKKTEVNLEAPEPGVEVIFYLSDREPLRLGSGEYTAEELCIRAAQACRISPLCHNLFALYDENTKLWYAPNRTITVDDKMSLRLHYRMRFYFTNWHGTNDNEQSVWRHSPKKQKNGYEKKKIPDATPLLDASSLEYLFAQGQYDLVKCLAPIRDPKTEQDGHDIENECLGMAVLAISHYAMMKKMQLPELPKDISYKRYIPETLNKSIRQRNLLTRMRINNVFKDFLKEFNNKTICDSSVSTHDLKVKYLATLETLTKHYGAEIFETSMLLISSENEMNWFHSNDGGNVLYYEVMVTGNLGIQWRHKPNVVSVEKEKNKLKRKKLENKHKKDEEKNKIREEWNNFSYFPEITHIVIKESVVSINKQDNKKMELKLSSHEEALSFVSLVDGYFRLTADAHHYLCTDVAPPLIVHNIQNGCHGPICTEYAINKLRQEGSEEGMYVLRWSCTDFDNILMTVTCFEKSEQVQGAQKQFKNFQIEVQKGRYSLHGSDRSFPSLGDLMSHLKKQILRTDNISFMLKRCCQPKPREISNLLVATKKAQEWQPVYPMSQLSFDRILKKDLVQGEHLGRGTRTHIYSGTLMDYKDDEGTSEEKKIKVILKVLDPSHRDISLAFFEAASMMRQVSHKHIVYLYGVCVRDVENIMVEEFVEGGPLDLFMHRKSDVLTTPWKFKVAKQLASALSYLEDKDLVHGNVCTKNLLLAREGIDSECGPFIKLSDPGIPITVLSRQECIERIPWIAPECVEDSKNLSVAADKWSFGTTLWEICYNGEIPLKDKTLIEKERFYESRCRPVTPSCKELADLMTRCMNYDPNQRPFFRAIMRDINKLEEQNPDIVSEKKPATEVDPTHFEKRFLKRIRDLGEGHFGKVELCRYDPEGDNTGEQVAVKSLKPESGGNHIADLKKEIEILRNLYHENIVKYKGICTEDGGNGIKLIMEFLPSGSLKEYLPKNKNKINLKQQLKYAVQICKGMDYLGSRQYVHRDLAARNVLVESEHQVKIGDFGLTKAIETDKEYYTVKDDRDSPVFWYAPECLMQSKFYIASDVWSFGVTLHELLTYCDSDSSPMALFLKMIGPTHGQMTVTRLVNTLKEGKRLPCPPNCPDEVYQLMRKCWEFQPSNRTSFQNLIEGFEALLK

SEQ ID NO: 54 - protein, 1132 aa, Homo sapiens
MGMACLTMTEMEGTSTSSIYQNGDISGNANSMKQIDPVLQVYLYHSLGKSEADYLTFPSGEYVAEEICIAASKACGITPVYHNMFALMSETERIWYPPNHVFHIDESTRHNVLYRIRFYFPRWYCSGSNRAYRHGISRGAEAPLLDDFVMSYLFAQWRHDFVHGWIKVPVTHETQEECLGMAVLDMMRIAKENDQTPLAIYNSISYKTFLPKCIRAKIQDYHILTRKRIRYRFRRFIQQFSQCKATARNLKLKYLINLETLQSAFYTEKFEVKEPGSGPSGEEIFATIIITGNGGIQWSRGKHKESETLTEQDLQLYCDFPNIIDVSIKQANQEGSNESRVVTIHKQDGKNLEIELSSLREALSFVSLIDGYYRLTADAHHYLCKEVAPPAVLENIQSNCHGPISMDFAISKLKKAGNQTGLYVLRCSPKDFNKYFLTFAVERENVIEYKHCLITKNENEEYNLSGTKKNFSSLKDLLNCYQMETVRSDNIIFQFTKCCPPKPKDKSNLLVFRTNGVSDVPTSPTLQRPTHMNQMVFHKIRNEDLIFNESLGQGTFTKIFKGVRREVGDYGQLHETEVLLKVLDKAHRNYSESFFEAASMMSKLSHKHLVLNYGVCVCGDENILVQEFVKFGSLDTYLKKNKNCINILWKLEVAKQLAWAMHFLEENTLIHGNVCAKNILLIREEDRKTGNPPFIKLSDPGISITVLPKDILQERIPWVPPECIENPKNLNLATDKWSFGTTLWEICSGGDKPLSALDSQRKLQFYEDRHQLPAPKWAELANLINNCMDYEPDFRPSFRAIIRDLNSLFTPDYELLTENDMLPNMRIGALGFSGAFEDRDPTQFEERHLKFLQQLGKGNFGSVEMCRYDPLQDNTGEVVAVKKLQHSTEEHLRDFEREIEILKSLQHDNIVKYKGVCYSAGRRNLKLIMEYLPYGSLRDYLQKHKERIDHIKLLQYTSQICKGMEYLGTKRYIHRDLATRNILVENENRVKIGDFGLTKVLPQDKEYYKVKEPGESPIFWYAPESLTESKFSVASDVWSFGVVLYELFTYIEKSKSPPAEFMRMIGNDKQGQMIVFHLIELLKNNGRLPRPDGCPDEIYMIMTECWNNNVNQRPSFRDLALRVDQIRDNMAG

SEQ ID NO: 55 - protein, 1124 aa, Homo sapiens
MAPPSEETPLIPQRSCSLLSTEAGALHVLLPARGPGPPQRLSFSFGDHLAEDLCVQAAKASGILPVYHSLFALATEDLSCWFPPSHIFSVEDASTQVLLYRIRFYFPNWFGLEKCHRFGLRKDLASAILDLPVLEHLFAQHRSDLVSGRLPVGLSLKEQGECLSLAVLDLARMAREQAQRPGELLKTVSYKACLPPSLRDLIQGLSFVTRRRIRRTVRRALRRVAACQADRHSLMAKYIMDLERLDPAGAAETFHVGLPGALGGHDGLGLLRVAGDGGIAWTQGEQEVLQPFCDFPEIVDISIKQAPRVGPAGEHRLVTVTRTDNQILEAEFPGLPEALSFVALVDGYFRLTTDSQHFFCKEVAPPRLLEEVAEQCHGPITLDFAINKLKTGGSRPGSYVLRRSPQDFDSFLLTVCVQNPLGPDYKGCLIRRSPTGTFLLVGLSRPHSSLRELLATCWDGGLHVDGVAVTLTSCCIPRPKEKSNLIVVQRGHSPPTSSLVQPQSQYQLSQMTFHKIPADSLEWHENLGHGSFTKIYRGCRHEVVDGEARKTEVLLKVMDAKHKNCMESFLEAASLMSQVSYRHLVLLHGVCMAGDSTMVQEFVHLGAIDMYLRKRGHLVPASWKLQVVKQLAYALNYLEDKGLPHGNVSARKVLLAREGADGSPPFIKLSDPGVSPAVLSLEMLTDRIPWVAPECLREAQTLSLEADKWGFGATVWEVFSGVTMPISALDPAKKLQFYEDRQQLPAPKWTELALLIQQCMAYEPVQRPSFRAVIRDLNSLISSDYELLSDPTPGALAPRDGLWNGAQLYACQDPTIFEERHLKYISQLGKGNFGSVELCRYDPLGDNTGALVAVKQLQHSGPDQQRDFQREIQILKALHSDFIVKYRGVSYGPGRQSLRLVMEYLPSGCLRDFLQRHRARLDASRLLLYSSQICKGMEYLGSRRCVHRDLAARNILVESEAHVKIADFGLAKLLPLDKDYYVVREPGQSPIFWYAPESLSDNIFSRQSDVWSFGVVLYELFTYCDKSCSPSAEFLRMMGCERDVPALCRLLELLEEGQRLPAPPACPAEVHELMKLCWAPSPQDRPSFSALGPQLDMLWSGSRGCETHAFTAHPEGKHHSLSFS

SEQ ID NO: 56 - protein, 1187 aa, Homo sapiens
MPLRHWGMARGSKPVGDGAQPMAAMGGLKVLLHWAGPGGGEPWVTFSESSLTAEEVCIHIAHKVGITPPCFNLFALFDAQAQVWLPPNHILEIPRDASLMLYFRIRFYFRNWHGMNPREPAVYRCGPPGTEASSDQTAQGMQLLDPASFEYLFEQGKHEFVNDVASLWELSTEEEIHHFKNESLGMAFLHLCHLALRHGIPLEEVAKKTSFKDCIPRSFRRHIRQHSALTRLRLRNVFRRFLRDFQPGRLSQQMVMVKYLATLERLAPRFGTERVPVCHLRLLAQAEGEPCYIRDSGVAPTDPGPESAAGPPTHEVLVTGTGGIQWWPVEEEVNKEEGSSGSSGRNPQASLFGKKAKAHKAVGQPADRPREPLWAYFCDFRDITHVVLKEHCVSIHRQDNKCLELSLPSRAAALSFVSLVDGYFRLTADSSHYLCHEVAPPRLVMSIRDGIHGPLLEPFVQAKLRPEDGLYLIHWSTSHPYRLILTVAQRSQAPDGMQSLRLRKFPIEQQDGAFVLEGWGRSFPSVRELGAALQGCLLRAGDDCFSLRRCCLPQPGETSNLIIMRGARASPRTLNLSQLSFHRVDQKEITQLSHLGQGTRTNVYEGRLRVEGSGDPEEGKMDDEDPLVPGRDRGQELRVVLKVLDPSHHDIALAFYETASLMSQVSHTHLAFVHGVCVRGPENIMVTEYVEHGPLDVWLRRERGHVPMAWKMVVAQQLASALSYLENKNLVHGNVCGRNILLARLGLAEGTSPFIKLSDPGVGLGALSREERVERIPWLAPECLPGGANSLSTAMDKWGFGATLLEICFDGEAPLQSRSPSEKEHFYQRQHRLPEPSCPQLATLTSQCLTYEPTQRPSFRTILRDLTRLQPHNLADVLTVNPDSPASDPTVFHKRYLKKIRDLGEGHFGKVSLYCYDPTNDGTGEMVAVKALKADCGPQHRSGWKQEIDILRTLYHEHIIKYKGCCEDQGEKSLQLVMEYVPLGSLRDYLPRHSIGLAQLLLFAQQICEGMAYLHAQHYIHRDLAARNVLLDNDRLVKIGDFGLAKAVPEGHEYYRVREDGDSPVFWYAPECLKEYKFYYASDVWSFGVTLYELLTHCDSSQSPPTKFLELIGIAQGQMTVLRLTELLERGERLPRPDKCPCEVYHLMKNCWETEASFRPTFENLIPILKTVHEKYQGQAPSVFSVC

SEQ ID NO: 57 - protein, 906 aa, Homo sapiens
MAGNVKKSSGAGGGSGSGGSGSGGLIGLMKDAFQPHHHHHHHLSPHPPGTVDKKMVEKCWKLMDKVVRLCQNPKLALKNSPPYILDLLPDTYQHLRTILSRYEGKMETLGENEYFRVFMENLMKKTKQTISLFKEGKERMYEENSQPRRNLTKLSLIFSHMLAELKGIFPSGLFQGDTFRITKADAAEFWRKAFGEKTIVPWKSFRQALHEVHPISSGLEAMALKSTIDLTCNDYISVFEFDIFTRLFQPWSSLLRNWNSLAVTHPGYMAFLTYDEVKARLQKFIHKPGSYIFRLSCTRLGQWAIGYVTADGNILQTIPHNKPLFQALIDGFREGFYLFPDGRNQNPDLTGLCEPTPQDHIKVTQEQYELYCEMGSTFQLCKICAENDKDVKIEPCGHLMCTSCLTSWQESEGQGCPFCRCEIKGTEPIVVDPFDPRGSGSLLRQGAEGAPSPNYDDDDDERADDTLFMMKELAGAKVERPPSPFSMAPQASLPPVPPRLDLLPQRVCVPSSASALGTASKAASGSLHKDKPLPVPPTLRDLPPPPPPDRPYSVGAESRPQRRPLPCTPGDCPSRDKLPPVPSSRLGDSWLPRPIPKVPVSAPSSSDPWTGRELTNRHSLPFSLPSQMEPRPDVPRLGSTFSLDTSMSMNSSPLVGPECDHPKIKPSSSANAIYSLAARPLPVPKLPPGEQCEGEEDTEYMTPSSRPLRPLDTSQSSRACDCDQQIDSCTYEAMYNIQSQAPSITESSTFGEGNLAAAHANTGPEESENEDDGYDVPKPPVPAVLARRTLSDISNASSSFGWLSLDGDPTTNVTEGSQVPERPPKPFPRRINSERKAGSCQQGSGPAASAATASPQLSSEIENLMSQGYSYQDIQKALVIAQNNIEMAKNILREFVSISSPAHVAT

SEQ ID NO: 58 - protein, 465 aa, Homo sapiens
MIVFVRFNSSHGFPVEVDSDTSIFQLKEVVAKRQGVPADQLRVIFAGKELRNDWTVQNCDLDQQSIVHIVQRPWRKGQEMNATGGDDPRNAAGGCEREPQSLTRVDLSSSVLPGDSVGLAVILHTDSRKDSPPAGSPAGRSIYNSFYVYCKGPCQRVQPGKLRVQCSTCRQATLTLTQGPSCWDDVLIPNRMSGECQSPHCPGTSAEFFFKCGAHPTSDKETSVALHLIATNSRNITCITCTDVRSPVLVFQCNSRHVICLDCFHLYCVTRLNDRQFVHDPQLGYSLPCVAGCPNSLIKELHHFRILGEEQYNRYQQYGAEECVLQMGGVLCPRPGCGAGLLPEPDQRKVTCEGGNGLGCGFAFCRECKEAYHEGECSAVFEASGTTTQAYRVDERAAEQARWEAASKETIKKTTKPCPRCHVPVEKNGGCMHMKCPQPQCRLEWCWNCGCEWNRVCMGDHWFDV

SEQ ID NO: 59 - protein, 108 aa, Homo sapiens [sequence omitted]

SEQ ID NO: 60 - protein, 501 aa, Homo sapiens
MAAASVTPPGSLELLQPGFSKTLLGTKLEAKYLCSACRNVLRRPFQAQCGHRYCSFCLASILSSGPQNCAACVHEGIYEEGISILESSSAFPDNAARREVESLPAVCPSDGCTWKGTLKEYESCHEGRCPLMLTECPACKGLVRLGEKERHLEHECPERSLSCRHCRAPCCGADVKAHHEVCPKFPLTCDGCGKKKIPREKFQDHVKTCGKCRVPCRFHAIGCLETVEGEKQQEHEVQWLREHLAMLLSSVLEAKPLLGDQSHAGSELLQRCESLEKKTATFENIVCVLNREVERVAMTAEACSRQHRLDQDKIEALSSKVQQLERSIGLKDLAMADLEQKVLEMEASTYDGVFIWKISDFARKRQEAVAGRIPAIFSPAFYTSRYGYKMCLRIYLNGDGTGRGTHLSLFFVVMKGPNDALLRWPFNQKVTLMLLDQNNREHVIDAFRPDVTSSSFQRPVNDMNIASGCPLFCPVSKMEAKNSYVRDDAIFIKAIVDLTGL

SEQ ID NO: 61 - protein, 491 aa, Homo sapiens
MCNTNMSVPTDGAVTTSQIPASEQETLVRPKPLLLKLLKSVGAQKDTYTMKEVLFYLGQYIMTKRLYDEKQQHIVYCSNDLLGDLFGVPSFSVKEHRKIYTMIYRNLVVVNQQESSDSGTSVSENRCHLEGGSDQKDLVQELQEEKPSSSHLVSRPSTSSRRRAISETEENSDELSGERQRKRHKSDSISLSFDESLALCVIREICCERSSSSESTGTPSNPDLDAGVSEHSGDWLDQDSVSDQFSVEFEVESLDSEDYSLSEEGQELSDEDDEVYQVTVYQAGESDTDSFEEDPEISLADYWKCTSCNEMNPPLPSHCNRCWALRENWLPEDKGKDKGEISEKAKLENSTQAEEGFDVPDCKKTIVNDSRESCVEENDDKITQASQSQESEDYSQPSTSSSIIYSSQEDVKEFEREETQDKEESVESSLPLNAIEPCVICQGRPKNGCIVHGKTGHLMACFTCAKKLKKRNKPCPVCRQPIQMIVLTYFP

SEQ ID NO: 62 - protein, 240 aa, Renilla reniformis
MTSKVYDPEQRKRMITGPQWWARCKQMNVLDSFINYYDSEKHAENAVIFLHGNAASSYLWRHVVPHIEPVARCIIPDLIGMGKSGKSGNGSYRLLDHYKYLTAWFELLNLPKKIIFVGHDWGACLAFHYSYEHQDKIKAIVHAESVVDVIESWDEWPDIEEDIALIKSEEGEKMVLENNFFVETMLPSKIMRKLEPEEFAAYLEPFKEKGEVRRPTLSWPREIPLVKGGKPDVVQIVRNY

SEQ ID NO: 63 - protein, 320 aa, Homo sapiens
MELLSPPLRDVDLTAPDGSLCSFATTDDFYDDPCFDSPDLRFFEDLDPRLMHVGALLKPEEHSHFPAAVHPAPGAREDEHVRAPSGHHQAGRCLLWACKACKRKTTNADRRKAATMRERRRLSKVNEAFETLKRCTSSNPNQRLPKVEILRNAIRYIEGLQALLRDQDAAPPGAAAAFYAPGPLPPGRGGEHYSGDSDASSPRSNCSDGMMDYSGPPSGARRRNCYEGAYYNEAPSEPRPGKSAAVSSLDCLSSIVERISTESPAAPALLLADVPSESPPRRQEAAAPSEGESSGDPTQSPDAAPQCPAGANPNPIYQVL

SEQ ID NO: 64 - protein, 607 aa, Homo sapiens
MAEKRRGSPCSMLSLKAHAFSVEALIGAEKQQQLQKKRRKLGAEEAAGAVDDGGCSRGGGAGEKGSSEGDEGAALPPPAGATSGPARSGADLERGAAGGCEDGFQQGASPLASPGGSPKGSPARSLARPGTPLPSPQAPRVDLQGAELWKRFHEIGTEMIITKAGRRMFPAMRVKISGLDPHQQYYIAMDIVPVDNKRYRYVYHSSKWMVAGNADSPVPPRVYIHPDSPASGETWMRQVISFDKLKLTNNELDDQGHIILHSMHKYQPRVHVIRKDCGDDLSPIKPVPSGEGVKAFSFPETVFTTVTAYQNQQITRLKIDRNPFAKGFRDSGRNRMGLEALVESYAFWRPSLRTLTFEDIPGIPKQGNASSSTLLQGTGNGVPATHPHLLSGSSCSSPAFHLGPNTSQLCSLAPADYSACARSGLTLNRYSTSLAETYNRLTNQAGETFAPPRTPSYVGVSSSTSVNMSMGGTDGDTFSCPQTSLSMQISGMSPQLQYIMPSPSSNAFATNQTHQGSYNTFRLHSPCALYGYNFSTSPKLAASPEKIVSSQGSFLGSSPSGTMTDRQMLPPVEGVHLLSSGGQQSFFDSRTLGSLTLSSSQVSAHMV

SEQ ID NO: 65 - protein, 393 aa, Homo sapiens [sequence omitted]

SEQ ID NO: 66 - protein, 215 aa, Homo sapiens
MGKGDPKKPRGKMSSYAFFVQTCREEHKKKHPDASVNFSEFSKKCSERWKTMSAKEKGKFEDMAKADKARYEREMKTYIPPKGETKKKFKDPNAPKRPPSAFFLFCSEYRPKIKGEHPGLSIGDVAKKLGEMWNNTAADDKQPYEKKAAKLKEKYEKDIAAYRAKGKPDAAKKGVVKAEKSKKKKEEEEDEEDEEDEEEEEDEEDEDEEEDDDDE

SEQ ID NO: 67 - protein, 356 aa, Homo sapiens
MTKSYSESGLMGEPQPQGPPSWTDECLSSQDEEHEADKKEDDLETMNAEEDSLRNGGEEEDEDEDLEEEEEEEEEDDDQKPKRRGPKKKKMTKARLERFKLRRMKANARERNRMHGLNAALDNLRKVVPCYSKTQKLSKIETLRLAKNYIWALSEILRSGKSPDLVSFVQTLCKGLSQPTTNLVAGCLQLNPRTFLPEQNQDMPPHLPTASASFPVHPYSYQSPGLPSPPYGTMDSSHVFHVKPPPHAYSAALEPFFESPLTDCTSPSFDGPLSPPLSINGNFSFKHEPSAEFEKNYAFTMHYPAATLAGAQSHGSIFSGTAAPRCEIPIDNIMSFDSHSHHERVMSAQLNAIFHD

SEQ ID NO: 68 - protein, 498 aa, Homo sapiens
MNQSIPVAPTPPRRVRLKPWLVAQVNSCQYPGLQWVNGEKKLFCIPWRHATRHGPSQDGDNTIFKAWAKETGKYTEGVDEADPAKWKANLRCALNKSRDFRLIYDGPRDMPPQPYKIYEVCSNGPAPTDSQPPEDYSFGAGEEEEEEEELQRMLPSLSLTEDVKWPPTLQPPTLRPPTLQPPTLQPPVVLGPPAPDPSPLAPPPGNPAGFRELLSEVLEPGPLPASLPPAGEQLLPDLLISPHMLPLTDLEIKFQYRGRPPRALTISNPHGCRLFYSQLEATQEQVELFGPISLEQVRFPSPEDIPSDKQRFYTNQLLDVLDRGLILQLQGQDLYAIRLCQCKVFWSGPCASAHDSCPNPIQREVKTKLFSLEHFLNELILFQKGQTNTPPPFEIFFCFGEEWPDRKPREKKLITVQVVPVAARLLLEMFSGELSWSADSIRLQISNPDLKDRMVEQFKELHHIWQSQQRLQPVAQAPPGAGLGVGQGPWPMHPAGMQ

SEQ ID NO: 69 - protein, 427 aa, Homo sapiens
MGTPKPRILPWLVSQLDLGQLEGVAWVNKSRTRFRIPWKHGLRQDAQQEDFGIFQAWAEATGAYVPGRDKPDLPTWKRNFRSALNRKEGLRLAEDRSKDPHDPHKIYEFVNSGVGDFSQPDTSPDTNGGGSTSDTQEDILDELLGNMVLAPLPDPGPPSLAVAPEPCPQPLRSPSLDNPTPFPNLGPSENPLKRLLVPGEEWEFEVTAFYRGRQVFQQTISCPEGLRLVGSEVGDRTLPGWPVTLPDPGMSLTDRGVMSYVRHVLSCLGGGLALWRAGQWLWAQRLGHCHTYWAVSEELLPNSGHGPDGEVPKDKEGGVFDLGPFIVDLITFTEGSGRSPRYALWFCVGESWPQDQPWTKRLVMVKVVPTCLRALVEMARVGGASSLENTVDLHISNSHPLSLTSDQYKAYLQDLVEGMDFQGPGES

SEQ ID NO: 70 - protein, 750 aa, Homo sapiens
MSQWYELQQLDSKFLEQVHQLYDDSFPMEIRQYLAQWLEKQDWEHAANDVSFATIRFHDLLSQLDDQYSRFSLENNFLLQHNIRKSKRNLQDNFQEDPIQMSMIIYSCLKEERKILENAQRFNQAQSGNIQSTVMLDKQKELDSKVRNVKDKVMCIEHEIKSLEDLQDEYDFKCKTLQNREHETNGVAKSDQKQEQLLLKKMYLMLDNKRKEVVHKIIELLNVTELTQNALINDELVEWKRRQQSACIGGPPNACLDQLQNWFTIVAESLQQVRQQLKKLEELEQKYTYEHDPITKNKQVLWDRTFSLFQQLIQSSFVVERQPCMPTHPQRPLVLKTGVQFTVKLRLLVKLQELNYNLKVKVLFDKDVNERNTVKGFRKFNILGTHTKVMNMEESTNGSLAAEFRHLQLKEQKNAGTRTNEGPLIVTEELHSLSFETQLCQPGLVIDLETTSLPVVVISNVSQLPSGWASILWYNMLVAEPRNLSFFLTPPCARWAQLSEVLSWQFSSVTKRGLNVDQLNMLGEKLLGPNASPDGLIPWTRFCKENINDKNFPFWLWIESILELIKKHLLPLWNDGCIMGFISKERERALLKDQQPGTFLLRFSESSREGAITFTWVERSQNGGEPDFHAVEPYTKKELSAVTFPDIIRNYKVMAAENIPENPLKYLYPNIDKDHAFGKYYSRPKEAPEPMELDGPKGTGYIKTELISVSEVHPSRLQTTDNLLPMSPEEFDEVSRIVGSVEFDSMMNTV

SEQ ID NO: 71 - protein, 225 aa, Homo sapiens
MVTHSKFPAAGMSRPLDTSLRLKTFSSKSEYQLVVNAVRKLQESGFYWSAVTGGEANLLLSAEPAGTFLIRDSSDQRHFFTLSVKTQSGTKNLRIQCEGGSFSLQSDPRSTQPVPRFDCVLKLVHHYMPPPGAPSFPSPPTEPSSEVPEQPSAQPLPGSPPRRAYYIYSGGEKIPLVLSRPLSSNVATLQHLCRKTVNGHLDSYEKVTQLPGPIREFLDQYDAPL

SEQ ID NO: 72 - protein, 770 aa, Homo sapiens
MAQWNQLQQLDTRYLEQLHQLYSDSFPMELRQFLAPWIESQDWAYAASKESHATLVFHNLLGEIDQQYSRFLQESNVLYQHNLRRIKQFLQSRYLEKPMEIARIVARCLWEESRLLQTAATAAQQGGQANHPTAAVVTEKQQMLEQHLQDVRKRVQDLEQKMKVVENLQDDFDFNYKTLKSQGDMQDLNGNNQSVTRQKMQQLEQMLTALDQMRRSIVSELAGLLSAMEYVQKTLTDEELADWKRRQQIACIGGPPNICLDRLENWITSLAESQLQTRQQIKKLEELQQKVSYKGDPIVQHRPMLEERIVELFRNLMKSAFVVERQPCMPMHPDRPLVIKTGVQFTTKVRLLVKFPELNYQLKIKVCIDKDSGDVAALRGSRKFNILGTNTKVMNMEESNNGSLSAEFKHLTLREQRCGNGGRANCDASLIVTEELHLITFETEVYHQGLKIDLETHSLPVVVISNICQMPNAWASILWYNMLTNNPKNVNFFTKPPIGTWDQVAEVLSWQFSSTTKRGLSIEQLTTLAEKLLGPGVNYSGCQITWAKFCKENMAGKGFSFWVWLDNIIDLVKKYILALWNEGYIMGFISKERERAILSTKPPGTFLLRFSESSKEGGVTFTWVEKDISGKTQIQSVEPYTKQQLNNMSFAEIIMGYKIMDATNILVSPLVYLYPDIPKEEAFGKYCRPESQEHPEADPGSAAPYLKTKFICVTPTTCSNTIDLPMSPRTLDSLMQFGNNGEGAEPSAGGQFESLTFDMELTSECATSPM

SEQ ID NO: 73 - protein, 246 aa, Homo sapiens
MELVLTQTPSPVSAVVGGTVTINCQSSQSVWGNNRLSWYQQKPGQPPRLLMYYASNLASGVSSRFKGSGSGTQFTLTISDVQCDDAATYYCQGGFECSGGDCVGFGGGTELEILGGSSRSSSSGGGGSGGGGQSVEESGGRLVAPGGSLTLTCTVSGIDLSSDAMSWVRQAPGKGLEWIGTIYGSAGTYYATWAKGRFTISKTSTTVDLKMTSLTTEDTATYFCTRAFSNTRLDLWGQGTLVTISS

SEQ ID NO: 74 - protein, 233 aa, Homo sapiens
MSQSNRELVVDFLSYKLSQKGYSWSQFSDVEENRTEAPEGTESEMETPSAINGNPSWHLADSPAVNGATGHSSSLDAREVIPMAAVKQALREAGDEFELRYRRAFSDLTSQLHITPGTAYQSFEQVVNELFRDGVNWGRIVAFFSFGGALCVESVDKEMQVLVSRIAAWMATYLNDHLEPWIQENGGWDTFVELYGNNAAAESRKGQERFNRWFLTGMTVAGVVLLGSLFSRK

SEQ ID NO: 75 - protein, 312 aa, Homo sapiens
MANNDAVLKRLEQKGAEADQIIEYLKQQVSLLKEKAILQATLREEKKLRVENAKLKKEIEELKQELIQAEIQNGVKQIPFPSGTPLHANSMVSENVIQSTAVTTVSSGTKEQIKGGTGDEKKAKEKIEKKGEKKEKKQQSIAGSADSKPIDVSRLDLRIGCIITARKHPDADSLYVEEVDVGEIAPRTVVSGLVNHVPLEQMQNRMVILLCNLKPAKMRGVLSQAMVMCASSPEKIEILAPPNGSVPGDRITFDAFPGEPDKELNPKKKIWEQIQPDLHTNDECVATYKGVPFEVKGKGVCRAQTMSNSGIK

SEQ ID NO: 76 - protein, 320 aa, Homo sapiens
MPMYQVKPYHGGGAPLRVELPTCMYRLPNVHGRSYGPAPGAGHVQEESNLSLQALESRQDDILKRLYELKAAVDGLSKMIQTPDADLDVTNIIQADEPTTLTTNALDLNSVLGKDYGALKDIVINANPASPPLSLLVLHRLLCEHFRVLSTVHTHSSVKSVPENLLKCFGEQNKKQPRQDYQLGFTLIWKNVPKTQMKFSIQTMCPIEGEGNIARFLFSLFGQKHNAVNATLIDSWVDIAIFQLKEGSSKEKAAVFRSMNSALGKSPWLAGNELTVADVVLWSVLQQIGGCSVTVPANVQRWMRSCENLAPFNTALKLLK

SEQ ID NO: 77 - protein, 233 aa, Homo sapiens
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDE

SEQ ID NO: 78 - protein, 267 aa, Homo sapiens
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKGSGSGLRSRAQASNSAVDGTAGPGSTGS

SEQ ID NO: 79 - protein, 425 aa, Homo sapiens
MADKEAAFDDAVEERVINEEYKIWKKNTPFLYDLVMTHALEWPSLTAQWLPDVTRPEGKDFSIHRLVLGTHTSDEQNHLVIASVQLPNDDAQFDASHYDSEKGEFGGFGSVSGKIEIEIKINHEGEVNRARYMPQNPCIIATKTPSSDVLVFDYTKHPSKPDPSGECNPDLRLRGHQKEGYGLSWNPNLSGHLLSASDDHTICLWDISAVPKEGKVVDAKTIFTGHTAVVEDVSWHLLHESLFGSVADDQKLMIWDTRSNNTSKPSHSVDAHTAEVNCLSFNPYSEFILATGSADKTVALWDLRNLKLKLHSFESHKDEIFQVQWSPHNETILASSGTDRRLNVWDLSKIGEEQSPEDAEDGPPELLFIHGGHTAKISDFSWNPNEPWVICSVSEDNIMQVWQMAENIYNDEDPEGSVDPEGQGS

SEQ ID NO: 80 - protein, 375 aa, Homo sapiens
MADHSFSDGVPSDSVEAAKNASNTEKLTDQVMQNPRVLAALQERLDNVPHTPSSYIETLPKAVKRRINALKQLQVRCAHIEAKFYEEVHDLERKYAALYQPLFDKRREFITGDVEPTDAESEWHSENEEEEKLAGDMKSKVVVTEKAAATAEEPDPKGIPEFWFTIFRNVDMLSELVQEYDEPILKHLQDIKVKFSDPGQPMSFVLEFHFEPNDYFTNSVLTKTYKMKSEPDKADPFSFEGPEIVDCDGCTIDWKKGKNVTVKTIKKKQKHKGRGTVRTITKQVPNESFFNFFNPLKASGDGESLDEDSEFTLASDFEIGHFFRERIVPRAVLYFTGEAIEDDDNFEEGEEGEEEELEGDEEGEDEDDAEINPKV

SEQ ID NO: 81 - protein, 1132 aa, Homo sapiens
MPRAPRCRAVRSLLRSHYREVLPLATFVRRLGPQGWRLVQRGDPAAFRALVAQCLVCVPWDARPPPAAPSFRQVSCLKELVARVLQRLCERGAKNVLAFGFALLDGARGGPPEAFTTSVRSYLPNTVTDALRGSGAWGLLLRRVGDDVLVHLLARCALFVLVAPSCAYQVCGPPLYQLGAATQARPPPHASGPRRRLGCERAWNHSVREAGVPLGLPAPGARRRGGSASRSLPLPKRPRRGAAPEPERTPVGQGSWAHPGRTRGPSDRGFCVVSPARPAEEATSLEGALSGTRHSHPSVGRQHHAGPPSTSRPPRPWDTPCPPVYAETKHFLYSSGDKEQLRPSFLLSSLRPSLTGARRLVETIFLGSRPWMPGTPRRLPRLPQRYWQMRPLFLELLGNHAQCPYGVLLKTHCPLRAAVTPAAGVCAREKPQGSVAAPEEEDTDPRRLVQLLRQHSSPWQVYGFVRACLRRLVPPGLWGSRHNERRFLRNTKKFISLGKHAKLSLQELTWKMSVRDCAWLRRSPGVGCVPAAEHRLREEILAKFLHWLMSVYVVELLRSFFYVTETTFQKNRLFFYRKSVWSKLQSIGIRQHLKRVQLRELSEAEVRQHREARPALLTSRLRFIPKPDGLRPIVNMDYVVGARTFRREKRAERLTSRVKALFSVLNYERARRPGLLGASVLGLDDIHRAWRTFVLRVRAQDPPPELYFVKVDVTGAYDTIPQDRLTEVIASIIKPQNTYCVRRYAVVQKAAHGHVRKAFKSHVSTLTDLQPYMRQFVAHLQETSPLRDAVVIEQSSSLNEASSGLFDVFLRFMCHHAVRIRGKSYVQCQGIPQGSILSTLLCSLCYGDMENKLFAGIRRDGLLLRLVDDFLLVTPHLTHAKTFLRTLVRGVPEYGCVVNLRKTVVNFPVEDEALGGTAFVQMPAHGLFPWCGLLLDTRTLEVQSDYSSYARTSIRASLTFNRGFKAGRNMRRKLFGVLRLKCHSLFLDLQVNSLQTVCTNIYKILLLQAYRFHACVLQLPFHQQVWKNPTFFLRVISDTASLCYSILKAKNAGMSLGAKGAAGPLPSEAVQWLCHQAFLLKLTRHRVTYVPLLGSLRTAQTQLSRKLPGTTLTALEAAANPALPSDFKTILD

SEQ ID NO: 82 - protein, 462 aa, Homo sapiens
METEQPEETFPNTETNGEFGKRPAEDMEEEQAFKRSRNTDEMVELRILLQSKNAGAVIGKGGKNIKALRTDYNASVSVPDSSGPERILSISADIETIGEILKKIIPTLEEGLQLPSPTATSQLPLESDAVECLNYQHYKGSDFDCELRLLIHQSLAGGIIGVKGAKIKELRENTQTTIKLFQECCPHSTDRVVLIGGKPDRVVECIKIILDLISESPIKGRAQPYDPNFYDETYDYGGFTMMFDDRRGRPVGFPMRGRGGFDRMPPGRGGRPMPPSRRDYDDMSPRRGPPPPPPGRGGRGGSRARNLPLPPPPPPRGGDLMAYDRRGRPGDRYDGMVGFSADETWDSAIDTWSPSEWQMAYEPQGGSGYDYSYAGGRGSYGDLGGPIITTQVTIPKDLAGSIIGKGGQRIKQIRHESGASIKIDEPLEGSEDRIITITGTQDQIQNAQYLLQNSVKQYSGKF

SEQ ID NO: 83 - protein, 745 aa, Homo sapiens
MERPPGLRPGAGGPWEMRERLGTGGFGNVCLYQHRELDLKIAIKSCRLELSTKNRERWCHEIQIMKKLNHANVVKACDVPEELNILIHDVPLLAMEYCSGGDLRKLLNKPENCCGLKESQILSLLSDIGSGIRYLHENKIIHRDLKPENIVLQDVGGKIIHKIIDLGYAKDVDQGSLCTSFVGTLQYLAPELFENKPYTATVDYWSFGTMVFECIAGYRPFLHHLQPFTWHEKIKKKDPKCIFACEEMSGEVRFSSHLPQPNSLCSLVVEPMENWLQLMLNWDPQQRGGPVDLTLKQPRCFVLMDHILNLKIVHILNMTSAKIISFLLPPDESLHSLQSRIERETGINTGSQELLSETGISLDPRKPASQCVLDGVRGCDSYMVYLFDKSKTVYEGPFASRSLSDCVNYIVQDSKIQLPIIQLRKVWAEAVHYVSGLKEDYSRLFQGQRAAMLSLLRYNANLTKMKNTLISASQQLKAKLEFFHKSIQLDLERYSEQMTYGISSEKMLKAWKEMEEKAIHYAEVGVIGYLEDQIMSLHAEIMELQKSPYGRRQGDLMESLEQRAIDLYKQLKHRPSDHSYSDSTEMVKIIVHTVQSQDRVLKELFGHLSKLLGCKQKIIDLLPKVEVALSNIKEADNTVMFMQGKRQKEIWHLLKIACTQSSARSLVGSSLEGAVTPQTSAWLPPTSAEHDHSLSCVVTPQDGETSAQMIEENLNCLGHLSTIIHEANEEQGNSMMNLDWSWLTE

SEQ ID NO: 84 - protein, 968 aa, Homo sapiens
MAEDDPYLGRPEQMFHLDPSLTHTIFNPEVFQPQMALPTDGPYLQILEQPKQRGFRFRYVCEGPSHGGLPGASSEKNKKSYPQVKICNYVGPAKVIVQLVTNGKNIHLHAHSLVGKHCEDGICTVTAGPKDMVVGFANLGILHVTKKKVFETLEARMTEACIRGYNPGLLVHPDLAYLQAEGGGDRQLGDREKELIRQAALQQTKEMDLSVVRLMFTAFLPDSTGSFTRRLEPVVSDAIYDSKAPNASNLKIVRMDRTAGCVTGGEEIYLLCDKVQKDDIQIRFYEEEENGGVWEGFGDFSPTDVHRQFAIVFKTPKYKDINITKPASVFVQLRRKSDLETSEPKPFLYYPEIKDKEEVQRKRQKLMPNFSDSFGGGSGAGAGGGGMFGSGGGGGGTGSTGPGYSFPHYGFPTYGGITFHPGTTKSNAGMKHGTMDTESKKDPEGCDKSDDKNTVNLFGKVIETTEQDQEPSEATVGNGEVTLTYATGTKEESAGVQDNLFLEKAMQLAKRHANALFDYAVTGDVKMLLAVQRHLTAVQDENGDSVLHLAIIHLHSQLVRDLLEVTSGLISDDIINMRNDLYQTPLHLAVITKQEDVVEDLLRAGADLSLLDRLGNSVLHLAAKEGHDKVLSILLKHKKAALLLDHPNGDGLNAIHLAMMSNSLPCLLLLVAAGADVNAQEQKSGRTALHLAVEHDNISLAGCLLLEGDAHVDSTTYDGTTPLHIAAGRGSTRLAALLKAAGADPLVENFEPLYDLDDSWENAGEDEGVVPGTTPLDMATSWQVFDILNGKPYEPEFTSDDLLAQGDMKQLAEDVKLQLYKLLEIPDPDKNWATLAQKLGLGILNNAFRLSPAPSKTLMDNYEVSGGTVRELVEALRQMGYTEAIEVIQAASSPVKTTSQAHSLPLSPASTRQQIDELRDSDSVCDSGVETSFRKLSFTESLTSGASLLTLNKMPHDYGQEGPLEGKI

(19) United States
(12) Patent Application Publication
(10) Pub. No.: US 2025/0009845 A1
(43) Pub. Date: Jan. 9, 2025

(54) EXOSOMES FOR TARGET SPECIFIC DELIVERY AND METHODS FOR PREPARING AND DELIVERING THE SAME

(71) Applicants: ILIAS BIOLOGICS INC., DAEJEON (KR); ILIAS THERAPEUTICS, INC., NEW YORK, NY (US)

(72) Inventors: Chulhee CHOI, Daejeon (KR); Nambin YIM, Daejeon (KR); Hojun CHOI, Daejeon (KR); Kyungsun CHOI, Daejeon (KR); Seung-Wook RYU, Daejeon (KR)

(73) Assignees: ILIAS BIOLOGICS INC., DAEJEON (KR); ILIAS THERAPEUTICS, INC., NEW YORK, NY (US)

(21) Appl. No.: 18/887,997

(22) Filed: Sep. 17, 2024

Related U.S. Application Data
(63) Continuation of application No. 18/412,403, filed on Jan. 12, 2024, now Pat. No. 12,133,879.
Application No. 18/412,403 is a continuation-in-part of application No. 16/887,029, filed on May 29, 2020, now Pat. No. 11,872,193.
Application No. 16/887,029 is a continuation of application No. 15/803,338, filed on Nov. 3, 2017, now Pat. No. 10,702,581.
Application No. 15/803,338 is a continuation-in-part of application No. PCT/KR2017/011070, filed on Sep. 30, 2017.
Application No. PCT/KR2017/011070 is a continuation-in-part of application No. PCT/KR2016/004750, filed on May 4, 2016.
Application No. 18/412,403 is a continuation-in-part of application No. 16/639,740, filed on Feb. 17, 2020, now abandoned.
(60) Provisional application No. 62/659,816, filed on Apr. 19, 2018.

(30) Foreign Application Priority Data
May 4, 2015 (KR) 10-2015-0062604
Aug. 27, 2015 (KR) 10-2015-0120934
Sep. 30, 2016 (KR) 10-2016-0126335
Sep. 30, 2016 (KR) 10-2016-0126921
Sep. 30, 2016 (KR) 10-2016-0126961
Oct. 4, 2016 (KR) 10-2016-0127486
Oct. 13, 2016 (KR) 10-2016-0132616
Feb. 10, 2017 (KR) 10-2017-0018637
Aug. 17, 2017 (KR) 10-2017-0104171

(51) Int. Cl. A61K 38/17; A61K 38/00; A61K 47/69; C07K 14/705; C07K 16/18; C07K 16/28; C12N 15/62; C12N 15/85; C12P 21/02

(52) U.S. Cl. CPC A61K 38/177; A61K 47/6917; C07K 14/705; C07K 14/70596; C07K 16/18; C07K 16/28; C12N 15/62; C12N 15/85; C12P 21/02; A61K 38/00; C07K 2317/80; C07K 2319/03; C07K 2319/70

(57) ABSTRACT

The present invention provides a method for producing an exosome that transfers an active substance specifically to a target and the exosome produced by the same; a method for delivering the active substance to the target tissue using the exosome; a pharmaceutical composition for delivery of the active substance comprising the exosome as an active ingredient; and a composition for preparing the exosome comprising an expression vector wherein the target peptide is inserted into an extracellular portion of a transmembrane protein.

### CROSS REFERENCE TO RELATED APPLICATIONS

This application claims benefit from Korean Patent Application No. 10-2017-0104171 filed Aug. 17, 2017, and U.S. Provisional Application No. 62/659,816 filed Apr. 19, 2018, the contents of each of which are incorporated herein by reference.

### SEQUENCE LISTING SUBMISSION VIA PATENT CENTER

A computer readable xml file, entitled “128742-5001-US-02_Sequence Listing_ST26.xml,” created on or about Mar. 12, 2024, with a file size of 109,707 bytes contains the sequence listing for this application and is hereby incorporated by reference in its entirety.

### FIELD OF THE INVENTION

The present invention relates to a method for preparing an exosome that delivers a substance in a target specific manner and an exosome prepared by the method.

### BACKGROUND OF THE INVENTION

The human body is composed of about 200 kinds of 100 trillion cells, in which the physiological activity is regulated by the action of various proteins to maintain life.

Cells are surrounded by membranes in bilayer structure composed of phospholipids, which block the entry of foreign substances into cells. Most of the protein drugs which have developed so far cannot pass through the cell membrane to enter the cell and can act on the outside of the cell or act on a receptor on the cell membrane to deliver the signal into the cell in order to show physiological effect.

Cytosol has lots of proteins which interact with each other to regulate physiological activity. So, if only a protein drug can be delivered inside the cell, that is, inside the cytosol, the cell activity would be controlled more effectively.

Recently, studies have been actively going on to establish a method for delivering a target protein directly into cells via cell membrane. When a recombinant protein of a target protein and protein transduction domains (PTDs), the peptide that passes through the cell membrane, is prepared and administered, it can enter the cytosol through the cell membrane. PTD is exemplified by HIV-1 TAT, HSV VP22, Antp, dfTAT, and Hph-1. A fusion protein prepared by combining the PTDs and a target protein is produced as a recombinant protein and at this time a separation process is required. However, this process is problematic in that the protein refolding is not performed properly, the activity is decreased, the protein is nonspecifically transferred, the risk of causing an immune reaction in vivo is large, the cost is high, and the yield is low.

On the other hand, a target protein combined with various nanoparticles such as Gold NP (nano particle), Liposome NP, Magnetic NP, and Polymeric NP can enter the cytoplasm through the cell membrane by endocytosis. However, most of the complexes of nanoparticles and target proteins are degraded in lysosomes in cells. If the target protein is degraded inside the lysosome, the activity of the protein is lost. Furthermore, it is difficult to separate the target protein and the nanoparticles in the cytoplasm, and the toxicity of the nanoparticles may be a problem as well.

Exosome is a small vesicle with a membrane structure in the size of 50^{˜}200 nm, which is secreted out of the cell with containing protein, DNA, and RNA for intercellular signaling.

Exosome was first found in the process of leaving only hemoglobin in the red blood cells by eliminating intracellular proteins at the last stage of red cell maturation. From the observation under electron microscope, it was confirmed that exosome is not separated directly from plasma membrane but discharged extracellular from the intracellular specific zone, called multivesicular bodies (MVBs). That is, when MVBs are fused with plasma membrane, such vesicles are discharged outside of the cell, which are called exosome.

It has not been clearly disclosed the molecular mechanism of the exosome generation. However, it is known that various immune cells including B-lymphocytes, T-lymphocytes, dendritic cells, megakaryocytes, and macrophages, stem cells, and tumor cells produce and secrete exosomes when they are alive.

Exosome contains various intracellular proteins, DNA, and RNA. These substances contained in the exosome secreted out of the cell and can be reintroduced into other cells by fusion or endocytosis and serve as intercellular messengers.

Exosomes with the desired protein inside can be used to treat various diseases in vivo. This requires efficient production of exosomes containing target proteins. Korean Patent Registration No. 10-0519384 discloses a method comprising:
1) the introduction of a gene for a specific antigen into a cell line;
2) stable expression of the protein produced from the introduced gene in the cell line; and
3) releasing it out of the cell through the exosome, and a method of using the produced exosome as a vaccine.

However, since the exosome is formed naturally in cells, even when a gene encoding a target protein is introduced into the production cells, the possibility of preparing the exosome containing the target protein is very low. There is a problem that the delivery efficiency of the exosome to the target tissue is low.

The tetraspanin family has four transmembrane domains, intracellular N- and C-termini and two extracellular loops protrude between the first and second, and third and fourth transmembrane domains.

CD9 is a 24-27 kD sized cell surface glycoprotein receptor belonging to the tetraspanin family, which regulates signal transduction actions important for regulating cell development, activity, growth and motility. In addition, it can regulate cell adhesion and cell migration and induces platelet activation involved in platelet-induced endothelial cell proliferation. In addition, it promotes muscle cell fusion and contributes to the maintenance of root canal.

The present invention provides a method for producing an exosome for target specific delivery comprising: preparing an expression vector by inserting a target peptide into an extracellular membrane domain of a transmembrane protein of an exosome; and producing the exosome comprising the target peptide located at the exosome membrane. Further, the present invention shows that the inserted target peptide is well expressed in HEK293T cells and that an active substance trapped in the exosome is well transferred into a target tissue.

### SUMMARY OF THE INVENTION

A certain embodiment of the present invention provides a method for producing the exosome that transfers the active substance specifically to the target tissue and the exosome produced by the same.

Another embodiment of the present invention provides a method for delivering the active substance to the target tissue using the exosome.

Still another embodiment of the present invention provides a pharmaceutical composition for the delivery of an active substance comprising the exosome as an active ingredient.

Still another embodiment of the present invention provides an expression vector wherein the target peptide is inserted into the extracellular membrane domain of the transmembrane protein.

### BRIEF DESCRIPTION OF THE DRAWINGS

FIG. 1A is a schematic diagram of a pSF-CMV-CMV-Sbfl vector comprising a ClBN gene, an EGFP gene, and a target peptide inserted CD9 gene complex, and FIG. 1B is a brief diagram showing insertion location of the target peptide in the CD9 protein structure.

FIG. 2 is an image showing the expression of an Angiopeptin-2 peptide complex in HEK293T cells treated with the exosome comprising the Angiopeptin-2 peptide complex.

FIG. 3 is an image showing the expression of an ApoB peptide complex in HEK293T cells treated with the exosome comprising the ApoB peptide complex.

FIG. 4 is an image showing the expression of an ApoE peptide complex in HEK293T cells treated with the exosome comprising the ApoE peptide complex.

FIG. 5 is an image showing the expression of a VCAM-1 internalization sequence peptide complex in HEK293T cells treated with the exosome comprising the VCAM-1 internalization sequence peptide complex.

FIG. 6 shows a schematic diagram of a pSF-CMV-CMV-Sbfl vector comprising a Cre recombinase-CRY2 gene, the ClBN gene, the EGFP gene, and the target peptide inserted CD9 gene complex.

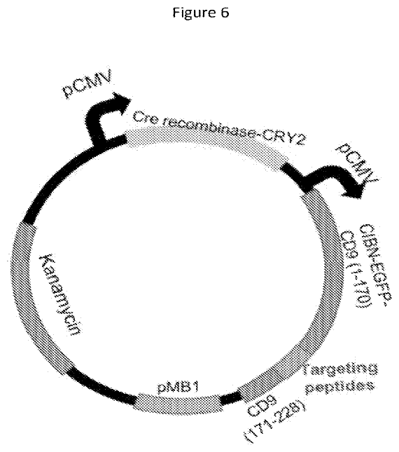

### DETAILED DESCRIPTION OF THE INVENTION

The present invention provides the method for producing the exosome that delivers the active substance specifically to the target tissue and the exosome produced by the same.

Another embodiment of the present invention provides the method for delivering the active substance to the target tissue using the exosome.

Still another embodiment of the present invention provides the pharmaceutical composition for the delivery of the active substance comprising the exosome as the active ingredient.

Still another embodiment of the present invention provides the expression vector wherein the target peptide is inserted into the extracellular membrane domain of the transmembrane protein.

The present invention relates to 1) the method for preparing the expression vector by inserting the target peptide into the extracellular membrane domain of the transmembrane protein of the exosome; and 2) the method for producing the exosome for target specific delivery of the active substance by introducing the said expression vector into an exosome-producing cell.

As used herein, the term “transmembrane protein” is a protein which locates and attached to the lipid bilayer of cells. It has hydrophobic regions containing a high fraction of polar amino acids. Certain hydrophobic regions locate inside the bilayer while more hydrophilic regions are in contact with the aqueous intracellular and extracellular environments. In one embodiment of the invention, the transmembrane protein is selected from the group such as, but not limited to tetraspanin, integrin, ICAM-1, MHC-I, MHC-II, annexin and Rab.

As used herein, the term “tetraspanin” is a membrane protein that has four transmembrane domains, presented on the cell membrane and receives information between cells and regulates cell proliferation. The tetraspanin is one or more proteins selected from the group comprising CD9, CD37, CD53, CD63, CD81 and CD82. In one embodiment of the invention, the tetraspanin is CD9.

The term “target peptide” as used herein, is a peptide capable of transferring a substance to a specific site in vivo. It is expressed on the surface of the exosome, allowing the exosome to migrate to the specific tissue. According to the present invention, any peptide able to migrate to the specific tissue can be used as the target peptide. In one embodiment of the invention, the target peptide is selected from but not limited to angiopeptin-2, ApoB, ApoE, VCAM-1 internalization sequence, striated muscle target peptide, Peptide-22, THR, THR retro-enantio, CTR, Leptin 20, RVG 29, CDX, Apamin, MiniAp-4, GSH, G23, g7, TGN, TAT (45-57), SynB1, Diketopeperazines and PhPro. The target peptide is inserted into the extracellular membrane domain of the transmembrane protein, wherein the insertion does not affect the expression or the function of the transmembrane. For example, the target peptide is inserted between amino acid position 170-171 from the N-terminus of the CD9 (SEQ ID NO: 3).

The term “specific site” as used herein, is the specific tissue where the target peptide migrates to. In one embodiment of the invention, the specific site is selected from but not limited to blood brain barrier, inflamed blood vessels, striated muscle, liver and cancer tissue.

The “expression vector” refers to a recombinant vector capable of expressing a desired peptide from a desired host cell, including an operatively linked necessary regulatory element to express the gene insert. The expression vector comprises expression control elements such as an initiation codon, a termination codon, a promoter, and an operator, etc. The initiation codon and the termination codon are generally considered as a nucleotide sequence and must be in frame with a coding sequence to encode a polypeptide. The promoter of the vector can be constitutive or inducible.

The term “operably linked” of the present invention means a functional linkage between a nucleic acid expression sequence and a nucleic acid sequence encoding a desired protein or RNA to perform a general function. For example, the expression of the coding sequence can be affected by operably linked a promoter and the protein or RNA coding nucleic sequence. The operable linkage with the expression vector can be produced by using recombinant DNA techniques well known in the art. A site-specific DNA cleavage and linkage can be achieved by using enzymes generally known in the art.

In addition, the expression vector may further includes a “selection marker”. Selection markers are markers for selection of a transformed microorganism or a recombinant vector which is used to confer selectable phenotypes, such as drug resistance, nutritional requirements, resistance to cytotoxic agents or expression of surface proteins. The transformed cells are selected using the vector containing the selection marker, as only the cells expressing the selection marker in the selected agent's environment can survive. The selection marker is selected from but not limited to the antibiotic resistance gene, for example kanamycin, ampicillin, and puromycin.

The “exosome-producing cell” is one or more selected from the group consisting of B-lymphocytes, T-lymphocytes, dendritic cells, macrophage cells, macrophages, stem cells, and tumor cells. In one embodiment of the invention, the exosome-producing cell is HEK293T cell.

As used herein, the term “active substance” refers to a substance that enhances or inhibits a biological function, wherein the active substance controls the secretion of substances that regulate the function of the human body exhibiting abnormal conditions. The active substance is selected from but not limited to a protein drug, an enzyme, a nucleic acid, a chemical and a mixture thereof.

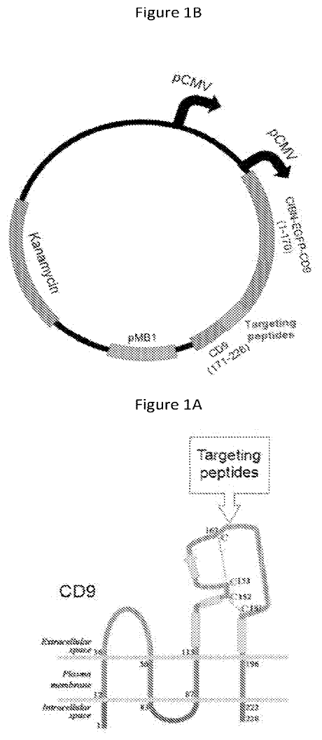

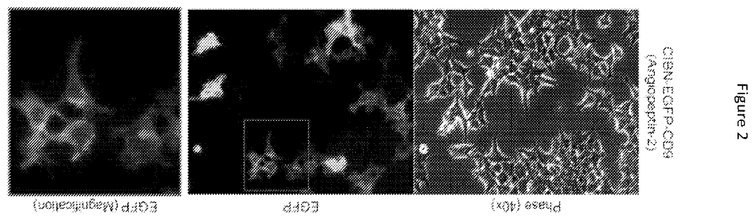

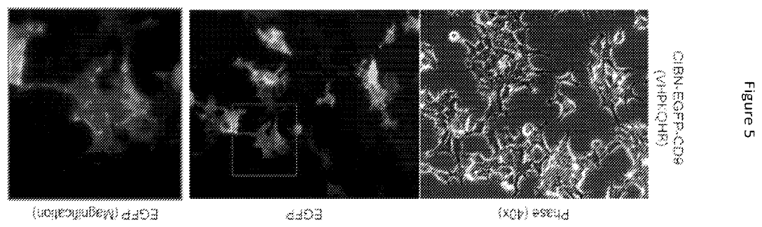

One embodiment of the present invention provides the pSF-CMV-CMV-Sbfl vector comprising the ClBN gene, the EGFP gene, and the target peptide complex inserted CD9 encoding gene, wherein the target peptide is selected from but not limited to angiopeptin-2, ApoB, ApoE, VCAM-1 internalization sequence, striated muscle target peptide, Peptide-22, THR, THR retro-enantio, CTR, Leptin 20, RVG 29, CDX, Apamin, MiniAp-4, GSH, G23, g7, TGN, TAT (45-57), SynB1, Diketopeperazines and PhPro. The said vector is introduced into exosome-producing cells such as HEK293T cells to obtain exosomes with target peptide labeled in the membrane protein (FIG. 1). FIGS. 2 and 5 show the expression of the target peptide in exosome membrane protein.

The present invention also provides the method for producing the exosome for target specific delivery of the active substance comprising:
1) preparing the expression vector by inserting the target peptide into the extracellular membrane domain of the transmembrane protein; and
2) introducing the expression vector of step 1) into the exosome-producing cell.

The transmembrane protein is selected from the group such as, but not limited to tetraspanin, integrin, ICAM-1, MHC-I, MHC-II, annexin and Rab. The tetraspanin is selected from the group consisting CD9, CD37, CD53, CD63, CD81 and CD82. In one embodiment of the invention, the tetraspanin is CD9.

The target peptide is any peptides able to migrate to the specific tissue. In one embodiment of the invention, the target peptide is selected from but not limited to angiopeptin-2, ApoB, ApoE, VCAM-1 internalization sequence, striated muscle target peptide, Peptide-22, THR, THR retro-enantio, CTR, Leptin 20, RVG 29, CDX, Apamin, MiniAp-4, GSH, G23, g7, TGN, TAT (45-57), SynB1, Diketopeperazines and PhPro.

The exosome-producing cell is one or more selected from the group comprising B-lymphocytes, T-lymphocytes, dendritic cells, macrophage cells, macrophages, stem cells, or tumor cells. In one embodiment of the invention, the exosome-producing cell is HEK293T cell.

In a specific embodiment of the present invention provides the pSF-CMV-CMV-Sbfl vector comprising the ClBN gene, the EGFP gene, and the target peptide complex inserted CD9 encoding gene, wherein the target peptide is selected from but not limited to angiopeptin-2, ApoB, ApoE, VCAM-1 internalization sequence and striated muscle target peptide. The said vector is introduced into exosome-producing cells such as HEK293T cells to obtain exosomes with target peptide labeled in the membrane protein (FIG. 1B). FIGS. 2 and 5 shows the expression of the target peptide in exosome membrane protein.

The present invention also provides the method for delivering the active substance to the target tissue using the exosome prepared by the method of the present invention.

The method comprises:
1) preparing the expression vector by inserting the target peptide into the extracellular membrane domain of the transmembrane protein; and
2) introducing the expression vector of step 1) into the exosome-producing cell.

The transmembrane protein is selected from the group such as, but not limited to tetraspanin, integrin, ICAM-1, MHC-I, MHC-II, annexin and Rab. The tetraspanin is selected from the group consisting CD9, CD37, CD53, CD63, CD81 and CD82. In one embodiment of the invention, the tetraspanin is CD9.

The target peptide is any peptides able to migrate to the specific tissue. In one embodiment of the invention, the target peptide is selected from but not limited to angiopeptin-2, ApoB, ApoE, VCAM-1 internalization sequence, striated muscle target peptide, Peptide-22, THR, THR retro-enantio, CTR, Leptin 20, RVG 29, CDX, Apamin, MiniAp-4, GSH, G23, g7, TGN, TAT (45-57), SynB1, Diketopeperazines and PhPro.

The exosome-producing cell is one or more selected from the group comprising B-lymphocytes, T-lymphocytes, dendritic cells, macrophage cells, macrophages, stem cells, or tumor cells. In one embodiment of the invention, the exosome-producing cell is HEK293T cell.

In a specific embodiment of the present invention provides the pSF-CMV-CMV-Sbfl vector comprising the ClBN gene, the EGFP gene, and the target peptide complex inserted CD9 encoding gene, wherein the target peptide is selected from but not limited to angiopeptin-2, ApoB, ApoE, VCAM-1 internalization sequence and striated muscle target peptide. The said vector is introduced into exosome-producing cells such as HEK293T cells to obtain exosomes with target peptide labeled in the membrane protein (FIG. 1B). FIGS. 2 and 5 shows the expression of the target peptide in exosome membrane protein.

The present invention also provides the pharmaceutical composition for the delivery of the active substance comprising the exosome as the active ingredient, wherein the amount of the exosome is about 10 to about 95% of the total weight of the composition.

The pharmaceutical composition of the present invention further comprises one or more active ingredients showing the same or similar functions to the above-mentioned active ingredient.

The pharmaceutical composition of the present invention further comprises pharmaceutically acceptable carriers, diluents, excipients and a mixture thereof. The pharmaceutically acceptable carrier is selected from but not limited to, chemicals listed in Merck Index, 13th ed., Merck & Co. Inc., saline solution, sterilized water, Ringer's solution, buffered saline, dextrose solution, maltodextrin solution, glycerol, ethanol and a mixture thereof. The pharmaceutical composition further comprises other conventional additives such as an antioxidant, a buffer, and a bacteriostatic agent.

The pharmaceutical composition further comprises a diluent or an excipient such as a filler, an extender, a binder, a wetting agent, a disintegrating agent, and a surfactant.

The pharmaceutical composition of the present invention is formulated into an oral or a parenteral preparation.

A solid formulation for the oral administration includes tablets, pills, powders, granules, capsules, troches and thereof. The solid formulation for the oral administration comprises one or more excipients such as starch, calcium carbonate, sucrose, lactose, gelatin, and thereof. The solid formulation further comprises lubricants such as magnesium stearate and talc.

A liquid formulation for the oral administration includes suspensions, solutions, emulsions, syrups and thereof. The liquid formulation comprises wetting agents, sweeteners, fragrances, preservatives and thereof.

The parenteral administration includes injections such as sterile aqueous solutions, non-aqueous solutions, suspensions, and emulsions. The non-aqueous solvent and the suspending agent is selected from the group comprising propylene glycol, polyethylene glycol, vegetable oil such as olive oil, injectable ester such as ethyl oleate, or thereof.

The pharmaceutical composition of the present invention is administered orally or parenterally according to the desired method. The parenteral administration is selected from external and intraperitoneal injection, intraperitoneal injection is selected from but not limited to rectal injection, subcutaneous injection, intravenous injection, and intramuscular injection.

The pharmaceutical composition according to the invention is administered in a pharmaceutically effective amount. The pharmaceutical effective amount varies on the type of disease, severity, activity of the drug, sensitivity to the drug, administration time, administration route, rate of excretion, duration of treatment, concurrent medication and thereof. The pharmaceutical composition of the present invention is administered alone or in combination with other therapeutic agents. When co-administered with other therapeutic agents, administration may be sequential or simultaneous.

The pharmaceutical composition of the present invention comprises the active ingredient wherein the pharmaceutically effective amount is 0.001-10 g/Kg, 0.01-8 g/Kg or 0.1-5 g/Kg. The administration can be once or several times a day.

In addition, the present invention provides the expression vector wherein the target peptide is inserted into the extracellular domain of the transmembrane protein.

The transmembrane protein is selected from the group such as, but not limited to tetraspanin, integrin, ICAM-1, MHC-I, MHC-II, annexin and Rab. The tetraspanin is one or more proteins selected from the group comprising CD9, CD37, CD53, CD63, CD81 or CD82. In one embodiment of the invention, the tetraspanin is CD9.

The target peptide is selected from but not limited to angiopeptin-2, ApoB, ApoE, VCAM-1 internalization sequence, striated muscle target peptide, Peptide-22, THR, THR retro-enantio, CTR, Leptin 20, RVG 29, CDX, Apamin, MiniAp-4, GSH, G23, g7, TGN, TAT (45-57), SynB1, Diketopeperazines and PhPro.

The expression vector is the recombinant vector capable of expressing the peptide of interest from the desired host cell, including the operatively linked necessary regulatory element to express the gene insert. The expression cells further comprise the selection marker. The selection marker is selected from but not limited to the antibiotic resistance gene, such as kanamycin, ampicillin, or puromycin. Any selection marker known in the art can be used.

The pharmaceutical composition may further comprises one or more other component compositions, solutions or devices suitable for the introduction of the expression vector, the culturing the transformed exosome producing cell, or the isolation and purification of the exosome produced from the transformed cells. For example, the composition further comprises a buffer suitable for the introduction of the expression vector, a medium and a container necessary for the culturing the transformed exosome producing cell and thereof.

An embodiment of the present invention provides the pSF-CMV-CMV-Sbfl vector comprising the ClBN gene, the EGFP gene, and the target peptide complex inserted CD9 encoding gene, wherein the target peptide is selected from but not limited to angiopeptin-2, ApoB, ApoE, VCAM-1 internalization sequence and striated muscle target peptide. The said vector is introduced into exosome-producing cells such as HEK293T cells to obtain exosomes with target peptide labeled in the membrane protein (FIG. 1). FIGS. 2 and 5 shows the expression of the target peptide in exosome membrane protein.

### EXAMPLE

Hereinafter, the present invention will be described in detail with reference to the following examples. However, the following examples are illustrative of the present invention, and the content of the present invention is not limited thereto.

### Example 1. Preparation of Exosomes Labeled with Angiopeptin-2 Peptide Complex in Exosomal Membrane Protein

Angiopeptin-2 is a protein targeting the blood-brain barrier. An exosome labeled with the Angiopeptin-2 peptide in the exosome membrane protein was prepared by the following method.

First, a multicloning site of pSF-CMV-CMV-Sbfl vector (#OG411, Oxford Genetics, UK), Ndel, was digested with Ndel restriction enzyme to linearize the DNA. Thereafter, the ClBN gene (SEQ ID NO: 1), the EGFP gene (SEQ ID NO: 2), a gene fragment of CD9 encoding 1-170 amino acids from the N-terminal, a gene fragment of CD9 encoding 171-228 amino acids from the N-terminal, and a gene fragment encoding the angiopeptin-2 peptide complex (SEQ ID NO: 4) was prepared by PCR. Next, the Ndel portion of the pSF-CMV-CMV-Sbfl vector was sequentially connected by Gibson assembly so that the two ends of the three fragments were overlapped with each other by 20 to 24 bp in order to obtain vector having a sequence of ClBN-EGFP-CD9 (1-170)-angiopeptin-2 peptide complex-CD9 (171-228). The angiopeptin-2 peptide complex is consisting with three repeated angiopeptin-2 amino acid sequences (SEQ ID NO: 5), and a linker described by the amino acid sequence of GGGGS (SEQ ID NO: 6) is located between angiopeptin-2 amino acid sequences, and a linker described in the amino acid sequence of PPVAT (SEQ ID NO: 7) is inserted at both ends of the angiopeptin-2 sequences.

The vector encoding ClBN-EGFP-CD9 (1-170)-angiopeptin 2 complex-CD9 (171-228) was introduced into HEK293T cells as exosome-producing cells. 24 hours incubation was followed by 48 hours incubation in the media without fetal bovine serum. The culture was centrifuged at 1,000 rpm for 3 minutes and was filtered using a polyethersulfone membrane having a pore size of 0.2 μm. The filtrate was first concentrated through tangential flow filtration at 4° C. The concentrate was then purified using size exclusion chromatography with a sepharose bead at 4° C. 300 to 500 ml of a phosphate buffered saline was added to dilute the solution, followed by secondary concentration through tangential flow filtration at 4° C. to obtained exosomes labeled with angiopeptin-2 peptide in the exosomal membrane.

### Example 2. Preparation of Exosomes Labeled with ApoB Peptide Complex in Exosomal Membrane

The ApoB is a protein targeting the blood-brain barrier, and the exosome labeled with the ApoB peptide complex in the exosomal membrane was prepared by the following method.

The same steps described in Example 1 were carried out, except only the ApoB peptide complex (SEQ ID NO: 8) was inserted to obtain the exosome labeled with the ApoB peptide complex in the exosomal membrane. The ApoB peptide complex is consisting with three repeated ApoB amino acid sequences (SEQ ID NO: 9), and the linker described by the amino acid sequence of GGGGS (SEQ ID NO: 6) is located between ApoB amino acid sequences, and the linker described in the amino acid sequence of PPVAT (SEQ ID NO: 7) is inserted at both ends of the ApoB sequences.

### Example 3. Preparation of Exosomes Labeled with ApoE Peptide Complex in Exosomal Membrane

The ApoE is a protein targeting the blood-brain barrier, and the exosome labeled with the ApoE peptide complex in exosomal membrane was prepared by the following method.

The same steps described in Example 1 were carried out, except only the ApoE peptide complex (SEQ ID NO: 10) was inserted to obtain the exosome labeled with the ApoE peptide complex in the exosomal membrane. The ApoE peptide complex is consisting with three repeated ApoE amino acid sequences (SEQ ID NO: 11), and the linker described by the amino acid sequence of GGGGS (SEQ ID NO: 6) is located between ApoE amino acid sequences, and the linker described in the amino acid sequence of PPVAT (SEQ ID NO: 7) is inserted at both ends of the ApoE sequences.

### Example 4. Production of Exosomes Labeled with VCAM-1 Internalization Sequence Peptide Complex in Exosomal Membrane

The VCAM-1 (vascular cell adhesion molecule-1) is a protein targeting the vascular inflammation site, and the exosome labeled with VCAM-1 internalization sequence peptide complex in the exosomal membrane was prepared by the following method.

The same steps described in Example 1 were carried out, except only the VCAM-1 internalization sequence peptide complex (SEQ ID NO: 12) was inserted to obtain the exosome labeled with the VCAM-1 internalization sequence peptide complex in the exosomal membrane. The VCAM-1 internalization sequence peptide complex is consisting with three repeated VCAM-1 internalization amino acid sequences (SEQ ID NO: 13), and the linker described by the amino acid sequence of GGGGS (SEQ ID NO: 6) is located between VCAM-1 internalization sequences, and the linker described in the amino acid sequence of PPVAT (SEQ ID NO: 7) is inserted at both ends of the VCAM-1 internalization sequences.

### Example 5. Preparation of Exosomes Labeled with Striated Muscle Target Peptide Complex in Exosomal Membrane

The striated muscle target peptide is a protein targeting striated muscle, and the exosome labeled with the straited muscle target peptide in the exosomal membrane was prepared by the following method.

The same steps described in Example 1 were carried out, except only the striated muscle target peptide complex (SEQ ID NOs: 14-16) was inserted to obtain the exosome labeled with the striated muscle target peptide complex in the exosomal membrane. Striated muscle target peptide complexes are consisting with three repeated amino acid sequence, ASSLNIA (SEQ ID NO: 17), TARGEHKEEELI (SEQ ID NO: 18) or SKTFNTHPQSTP (SEQ ID NO: 19), the linker described by the amino acid sequence of GGGGS (SEQ ID NO: 6) is located between sequences, and the linker described in the amino acid sequence of PPVAT (SEQ ID NO: 7) is inserted at both ends of the sequences.

### Example 6. Expression of Angiopopein-2 Peptide Complex

The exosome of Example 1 was transfected to HEK293T cells. The expression of the angioprotein-2 peptide complex in the exosomal membrane was confirmed through a fluorescence microscope after 24 hours. FIG. 2 shows the expression of the angioprotein-2 peptide complex in the exosomal membrane.

### Example 7. Expression of ApoB Peptide Complex

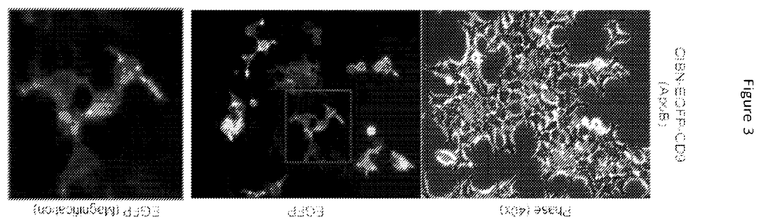

The exosome of Example 2 was transfected to HEK293T cells. The expression of the ApoB peptide complex in the exosomal membrane was confirmed through the fluorescence microscope after 24 hours. FIG. 3 shows the expression of the ApoB peptide complex in the exosomal membrane.

### Example 8. Expression of ApoE Peptide Complex

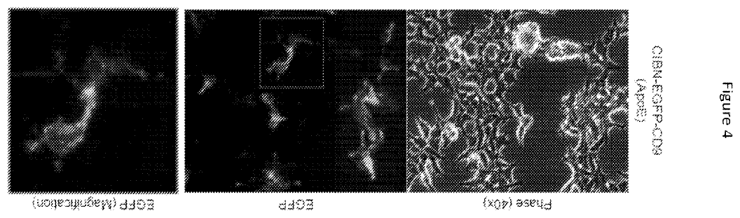

The exosome of Example 3 was transfected to HEK293T cells. The expression of the ApoE peptide complex in the exosomal membrane was confirmed through the fluorescence microscope after 24 hours. FIG. 4 shows the expression of the ApoE peptide complex in the exosomal membrane.

### Example 9. Expression of VCAM-1 Internalization Sequence Peptide Complex

The exosome of Example 4 was transfected to HEK293T cells. The expression of the VCAM-1 internalization sequence peptide complex in the exosomal membrane was confirmed through the fluorescence microscope after 24 hours. FIG. 5 shows the expression of the VCAM-1 internalization sequence peptide complex in the exosomal membrane.

### Example 10. Expression of Striated Muscle Target Peptide Complex

The exosome of Example 5 was transfected to HEK293T cells. The expression of the striated muscle target peptide complex in the exosomal membrane was confirmed through the fluorescence microscope after 24 hours. The expression of the striated muscle target peptide complex in the exosomal membrane was confirmed.

### Example 11. Target-Specific Delivery of Exosomes Labeled with Angiopeptin-2 Peptide Complex on Exosomal Membrane

The vector encoding ClBN-EGFP-CD9 (1-170)-angiopeptin 2 peptide complex-CD9 (171-228) was obtained with the same steps described in Example 1, except that an additional Cre recombinase-CRY2 gene was further inserted under an LED emitting light of 460 nm at an intensity of 100 μW. The vector was introduced to HEK293T as the exosome production cell. 24 hours incubation was followed by 48 hours incubation in the media without fetal bovine serum under the LED light. The culture medium was separated by tangential flow filtration and size exclusion chromatography to obtain exosomes labeled with the angiopeptin-2 peptide complex in the exosomal membrane. An exosome in which angiopeptin-2 peptide complex was not labeled on the exosomal membrane was used as a control group. The resulting exosome at a concentration of 1×10⁹ particles/50 μl was injected intravenously or intraperitoneally into the blood vessels of C57BL/6 loxP-eNphr3.0-loxP-eYFP TG mice (The Jackson Laboratory, Bar Harbor, Maine, USA) and organs were excised and histo-pathologically examined 48 or 72 hours after the injection. The distribution of eYFP in mice was analyzed to determine the function and distribution of the exosome labeled with the specific target peptide in vivo.

As a result, the exosome labeled with the angiopeptin-2 peptide was specifically transferred to the blood brain barrier.

### Example 12. Target-Specific Delivery Effect of Exosome Labeled with ApoB Peptide Complex in Exosomal Membrane

The vector encoding ClBN-EGFP-CD9 (1-170)-ApoB peptide complex-CD9 (171-228) was obtained the same steps described in Example 2, except that the additional Cre recombinase-CRY2 gene was further inserted under the LED emitting light of 460 nm at the intensity of 100 μW. Same steps described in Example 11 were carried out to determine the function and the distribution of the exosome labeled with the specific target peptide in vivo.

As a result, the exosome labeled with the ApoB peptide complex was specifically transferred to the blood brain barrier.

### Example 13. Target-Specific Delivery Effect of Exosome Labeled with ApoE Peptide Complex in Exosomal Membrane

The vector encoding ClBN-EGFP-CD9 (1-170)-ApoE peptide complex-CD9 (171-228) was obtained the same steps described in Example 3, except that the additional Cre recombinase-CRY2 gene was further inserted under the LED emitting light of 460 nm at the intensity of 100 μW. Same steps described in Example 11 were carried out to determine the function and the distribution of the exosome labeled with the specific target peptide in vivo.

As a result, the exosome labeled with the ApoE peptide complex was specifically transferred to the blood brain barrier.

### Example 14. Target-Specific Delivery Effect of Exosome Labeled with VCAM-1 Internalization Sequence Peptide Complex in Exosomal Membrane

The vector encoding ClBN-EGFP-CD9 (1-170)-VCAM-1 internalization sequence peptide complex-CD9 (171-228) was obtained the same steps described in Example 4, except that the additional Cre recombinase-CRY2 gene was further inserted under the LED emitting light of 460 nm at the intensity of 100 μW. Same steps described in Example 11 were carried out to determine the function and the distribution of the exosome labeled with the specific target peptide in vivo.

As a result, it was confirmed that the exosome labeled with the VCAM-1 internalization sequence peptide complex in the membrane protein was specifically transferred to the site of vascular inflammation.

### Example 15. Target-Specific Delivery Effect of Exosome Labeled with Striated Muscle Target Peptide Complex in Exosomal Membrane

The vector encoding ClBN-EGFP-CD9 (1-170)-striated muscle target peptide complex-CD9 (171-228) was obtained the same steps described in Example 5, except that the additional Cre recombinase-CRY2 gene was further inserted under the LED emitting light of 460 nm at the intensity of 100 μW. Same steps described in Example 11 were carried out to determine the function and the distribution of the exosome labeled with the specific target peptide in vivo.

As a result, it was confirmed that exosome labeled with the striated muscle target peptide complex in the membrane protein was specifically transferred to the striated muscle.

1. An exosome comprising
CD9, and
a target peptide inserted between amino acids corresponding to amino acid positions 167 and 181 from the N-terminus of the CD9.

2. The exosome of claim 1, wherein said CD9 comprises an amino acid sequence of SEQ ID NO: 3.

3. The exosome of claim 1, wherein said CD9 consists of an amino acid sequence of SEQ ID NO: 3.

4. The exosome of claim 2, wherein the target peptide is inserted between amino acid positions 167 and 181 from the N-terminus of the CD9.

5. The exosome of claim 1, wherein the target peptide is inserted between amino acids corresponding to amino acid positions 170 and 171 from the N-terminus of CD9,
and the target peptide is configured to transfer the exosome to a target tissue.

6. The exosome of claim 2, wherein the target peptide is inserted between amino acid positions 170 and 171 from the N-terminus of the CD9.

7. The exosome of claim 5, wherein the target tissue comprises at least one selected from the group consisting of blood brain barrier, inflamed blood vessels, striated muscle, liver, and cancer tissue.

8. The exosome of claim 1, wherein the target peptide comprises at least one selected from the group consisting of angiopep-2, ApoB, ApoE, VCAM-1 (vascular cell adhesion molecule-1) internalization sequence peptide complex, striated muscle target peptide, Peptide-22, THR, THR retro-enantio, CRT, Leptin30, RVG (Rabies Virus Glycoprotein) 29, CDX, Apamin, MiniAp-4, GSH, G23, g7, TGN, TAT (45-57), SynB1, Diketopiperazines, and PhPro.

9. The exosome of claim 1, wherein the target peptide does not affect expression or function of the CD9.

10. The exosome of claim 1, wherein the exosome further comprises one or more active substance(s) from the group consisting of a protein drug, an enzyme, a nucleic acid, and a chemical.

11. An exosome comprising
CD9, and
a target peptide inserted between amino acids corresponding to amino acid positions 167 and 181 from the N-terminus of SEQ ID NO:3.

12. The exosome of claim 11, wherein the target peptide is inserted between amino acid positions 167 and 181 from the N-terminus of SEQ ID NO:3.

13. The exosome of claim 11, wherein the target peptide is inserted between amino acid positions 170 and 171 from the N-terminus of SEQ ID NO:3.

14. A pharmaceutical composition comprising an exosome prepared of claim 1.

15. A method of producing the exosome of claim 1, comprising:
a) preparing an expression vector by inserting the target peptide between amino acids corresponding to amino acid positions 167 and 181 from the N-terminus of the CD9;
b) introducing the expression vector of the step a) into an exosome-producing cell; and
c) producing the exosome, wherein the target peptide is expressed on the surface of the exosome.

16. The method of claim 15, wherein said CD9 comprises an amino acid sequence of SEQ ID NO: 3.

17. The method of claim 15, wherein the target peptide is inserted between amino acid positions 167 and 181 from the N-terminus of the CD9.

18. The method of claim 15, wherein the target peptide is inserted between the amino acids corresponding to amino acid position 170 and 171 from the N-terminus of CD9,
and the target peptide is configured to transfer the exosome to a target tissue.

19. The method of claim 15, wherein the target peptide is inserted between amino acid positions 170 and 171 from the N-terminus of the CD9.

20. The method of claim 15, wherein the exosome-producing cell comprises at least one selected from the group consisting of B-lymphocytes, T-lymphocytes, dendritic cells, macrophage cells, macrophages, stem cells, HEK293T cells, and tumor cells.
